SEQUENCE LISTING (18 sequences)

SEQ ID NO: 1 - protein, 12 aa, synthetic construct
  REGION 1..12: note = Synthetic
  VAR_SEQ 12: note = note = Xaa - Xaa is Ala or Asp or Glu or Phe or Gly or His or Ile or Lys or Leu or Met or Asn or Pro or Gln or Arg or Ser or Thr or Val or Trp or Tyr
IWTKDHEEVYEX

SEQ ID NO: 2 - protein, 12 aa, synthetic construct
  REGION 1..12: note = Synthetic
IWTKDHEEVYEA

SEQ ID NO: 3 - protein, 30 aa, synthetic construct
  REGION 1..30: note = GLP-1
HAEGTFTSDVSSYLEGQAAKEFIAWLVKGR

SEQ ID NO: 4 - protein, 20 aa, synthetic construct
  REGION 1..20: note = PSTi8
PEGKGEQEHSQQKEEEEMAV

SEQ ID NO: 5 - protein, 34 aa, synthetic construct
  REGION 1..34: note = PYY(3-36)
IKPEAPGEDASPEELNRYYASLRHYLNLVTRQRY

SEQ ID NO: 6 - protein, 12 aa, synthetic construct
  REGION 1..12: note = LHRH
PYRHWSYGLRPG

SEQ ID NO: 7 - protein, 27 aa, synthetic construct
  REGION 1..27: note = Gastrin releasing peptide
VPLPAGGGTVLTKMYPRGNHWAVGHLM

SEQ ID NO: 8 - protein, 24 aa, synthetic construct
  REGION 1..24: note = Cortrosyn
SYSMEHFRWGKPVGKKRRPVKVYP

SEQ ID NO: 9 - protein, 43 aa, synthetic construct
  REGION 1..43: note = QRF-amide
QDEGSEATGFLPAAGEKTSGPLGNLAEELNGYSRKKGGFSFRF

SEQ ID NO: 10 - protein, 45 aa, synthetic construct
  REGION 1..45: note = EP45
HGEGTFTSDLSKQMEEEAVRLFIEWLKNGGPSSRHYLNLVTRQRY

SEQ ID NO: 11 - protein, 59 aa, synthetic construct
  REGION 1..59: note = Bulevertide
MYRISTICACIDGTNLSVPNPLGFFPDHQLDPAFGANSNNPDWDFNPNKDHWPEANKVG

SEQ ID NO: 12 - protein, 50 aa, synthetic construct
  REGION 1..50: note = Osteocrin
SFSGFGSPLDRLSAGSVDHKGKQRKVVDHPKRRFGIPMDRIGRNRLSNSR

SEQ ID NO: 13 - protein, 38 aa, synthetic construct
  REGION 1..38: note = Enfuvirtide
ACYTSLIHSLIEESQNQQEKNEQELLELDKWASLWNWF

SEQ ID NO: 14 - protein, 37 aa, synthetic construct
KCNTATCATQRLANFLVHSSNNFGPILPPTNVGSNTY

SEQ ID NO: 15 - protein, 20 aa, synthetic construct
IECTKSEGCEEVYEADHGEP

SEQ ID NO: 16 - protein, 66 aa, synthetic construct
EGTFTSDVSSYLEEQAAREFIAWLVRGRKGGGGEASELSTAALGRLSAELHELATLPRTETGSGSP

SEQ ID NO: 17 - protein, 80 aa, synthetic construct
DVKPGQPLEDEGDREGTFTSDVSSYLEEQAAREFIAWLVRGRKGGGGEASELSTAALGRLSAELHELATLPRTETGSGSP

SEQ ID NO: 18 - protein, 14 aa, synthetic construct
DVKPGQPLEDEGDR

(19) United States
(12) Patent Application Publication
(10) Pub. No.: US 2025/0042935 A1
(43) Pub. Date: Feb. 6, 2025

(54) PHOTOCHEMICAL APPROACH TO C-TERMINAL A-AMIDATION

(71) Applicant: Novo Nordisk A/S, Bagsvaerd (DK)

(72) Inventors: Michael Richard Harris, Seattle, WA (US); Sydnee Wong, Redmond, WA (US); David Thomas Hymel, Shoreline, WA (US); Wouter Frederik Johan Hogendorf, Oelstykke (DK); Felix Wojcik, Copenhagen (DK); Nicholas Raymond Cox, Brainbridge Island, WA (US); Benjamin Matthew Williams, Frederiksberg (DK); Asmus Ringlebjerg Mortensen, Vaerloese (DK)

(21) Appl. No.: 18/715,574

(22) PCT Filed: Dec. 9, 2022
(86) PCT No.: PCT/EP2022/085255

§ 371 Date: May 31, 2024

Related U.S. Application Data
(60) Provisional application No. 63/288,071, filed on Dec. 10, 2021.

(30) Foreign Application Priority Data
Jan. 20, 2022 (EP) 22152378.0

(51) Int. Cl. C07K 1/00; A61K 38/00

(52) U.S. Cl. CPC C07K 1/003; A61K 38/00

(57) ABSTRACT

The present invention relates to a photochemical process for the manufacturing of a C-terminal α-amide, which are a class of compounds that represent up to half of all biologically relevant peptide hormones. The invention provides mild, broad in scope, economically efficient process, which is suitable for large scale manufacturing.

### TECHNICAL FIELD OF THE INVENTION

The present invention relates to a photochemical process for the manufacturing of a C-terminal a-amide, and more specifically to a method of producing a peptide or protein comprising a C-terminal α-amide with a photochemical process.

### BACKGROUND

C-terminal α-amidation is a common post-translational modification that occurs in over half of all biologically active peptide hormones and neuropeptides and is often required for full biological activity.

Chemical C-terminal α-amidation requires selective functionalization of the C-terminus. Production of amidated peptides has been accomplished by fully synthetic approaches (Enzyme Microb. Technol. 16, 450-456 (1994)) and by in vitro enzymatic oxidation of recombinantly expressed C-terminal glycine extended precursors (Nat. Biotechnol. 11, 64-70 (1993)). However, these approaches are complex and expensive. For instance, peptidylglycine α-amidating monooxygenase (PAM) and related amidating enzymes are not only substrate specific but must be expressed in mammalian cultures, which adds cost to the manufacturing process.

Baker et al. provides a method for the selective modification of cysteines with bromomaleimides and purports to disclose a method for photolytic modification of cysteine maleimide conjugates (Chem. Commun. 6583-6585 (2009) and Org. Biomol. Chem. 14, 455-459 (2016)).

Enzymatic approaches to selective C-terminal functionalization and α-amidation that include a photolysis step are further set out in U.S. Pat. No. 5,580,751 and Int. J. Peptide Protein Res. 41, 169-180 (1993). Malins et al. allegedly discloses an electrochemical approach to selective C-terminal modification and α-amidation (J. Am. Chem. Soc. 143, 11811-11819 (2021)).

Yet, there remains a challenge in providing a low-cost, selective, and scalable process compatible with manufacturing-scale preparation of peptides containing a C-terminal α-amide. There always remains room for improvement.

### SUMMARY

One objective of the present work is to provide a photochemical process for the production of a peptide or protein comprising a C-terminal α-amide. It is an objective to provide a selective and low-cost C-terminal peptide and protein amidation process capable of being applied to a variety of peptides while overcoming at least some of the drawbacks of traditional amidation methods.

The overall photochemical reaction is depicted in Scheme 1 below and further depicted in FIG. 1. Disclosed herein is a method for producing a peptide or protein comprising a C-terminal α-amide of formula IV, following scheme 1:

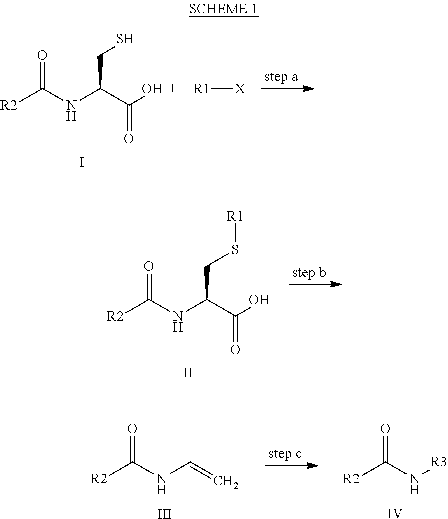

wherein R2 is a polypeptide,
R1-X is a photolabeling agent, wherein R1 is a photolabel and X is a leaving group,
R3 is selected from the group consisting of hydrogen, methyl and ethyl, comprising the steps of
   Step (a). coupling a peptide or protein comprising a C-terminal cysteine amidation tag (formula I) with the photolabel (R1) to obtain a peptide-photolabel conjugate (formula II);
   Step (b). irradiating the resulting peptide-photolabel conjugate (formula II) to obtain a C-terminal enamide (formula III) via a photochemical conversion; and
   Step (c). cleaving the C-terminal enamide (formula III) to obtain the C-terminal α-amide (formula IV).

In a first aspect of the invention, a method following Scheme 1 for producing a peptide or protein comprising a C-terminal α-amide of formula IV is provided. This is achieved by starting with a peptide or protein comprising a C-terminal cysteine amidation tag (depicted as formula I) and reacting it with a photolabeling agent (depicted as R1-X) to obtain a peptide-photolabel conjugate (formula II). Subsequently, the peptide-photolabel conjugate is exposed to light, and undergoes a photochemical conversion, to obtain a peptide or protein enamide (formula III), which is subsequently converted to the resulting C-terminal α-amide (formula IV).

The method for producing a peptide or protein comprising a C-terminal α-amide may comprise a photolabeling agent (R1-X) selected from the group consisting of 3-Bromo-1H-pyrrole-2,5-dione, 4-chloro-7-nitrobenzofurazan, 2-bromo-1,4-naphthoquinone, 1-fluoro-2,4-dinitrobenzene and 4-fluoro-7-sulfamoylbenzofurazan. The method for producing a peptide or protein comprising a C-terminal α-amide may comprise the use of cleavage reagent selected from the group consisting of trifluoroacetic acid (TFA), hydrochloric acid (HCl), Sulfuric acid (H₂SO₄), tosylic acid (TsOH), phosphoric acid (H₃PO₄), oxalic acid, 3,6-diphenyl-1,2,4,5-tetrazine, and 6,6′-(1,2,4,5-tetrazine-3,6-diyl)dinicotinic acid.

The method for producing a peptide or protein comprising a C-terminal α-amide may comprise a polypeptide R2 comprising an amino acid sequence as set out in any one of SEQ. ID No.: 3, 4, 5, 6, 7, 8, 9, 10, 11, 12, 13, 14, 16 or 17.

Disclosed is a method for producing a peptide or protein comprising a C-terminal α-amide, following Scheme 2 seen below and further depicted in FIG. 2,

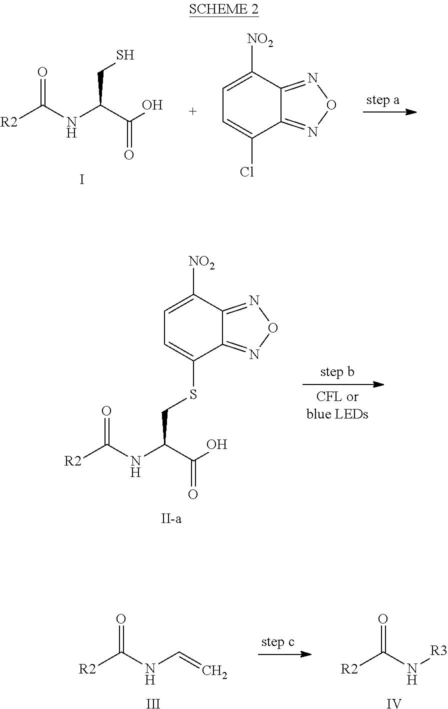

wherein R2 is a polypeptide,
R3 is hydrogen,
comprising the steps of
   Step (a). coupling a peptide or protein comprising a C-terminal cysteine amidation tag (formula I) with 4-chloro-7-nitrobenzofurazan to obtain a peptide-photolabel conjugate (formula II-a);
   Step (b). irradiating the peptide-photolabel conjugate (formula II-a) to obtain a C-terminal enamide (formula III) via photochemical conversion; and
   Step (c). cleaving the C-terminal enamide (formula III) to obtain the C-terminal α-amide (formula IV).

The invention according to the second aspect is carried out by reacting a peptide or protein comprising a C-terminal cysteine of formula I with 4-chloro-7-nitrobenzofurazan as the photolabeling agent, obtaining a peptide-photolabel conjugate of formula II-a. The conjugate is subsequently converted by a photochemical reaction to the enamide of formula III, which is converted by cleavage to the desired C-terminal α-amide of formula IV.

The method for producing a peptide or protein comprising a C-terminal α-amide may further comprise the steps of coupling a cysteine of the peptide R2 with the photolabel R1 forming a photolabel-protected cysteine prior to irradiation (step (b)); and releasing the photolabel-protected cysteine from the peptide R2 after irradiation (step (b)). The method may further comprise the step of adding a nucleophilic sulfide for releasing the photolabel-protected cysteine from the peptide R2.

The photochemical amidation method is mild, broad in scope, economically efficient, and suitable for large scale manufacturing. The invention may also solve further problems that will be apparent from the disclosure of the exemplary embodiments.

### SEQUENCE LISTING

SEQ ID No: 1 represents the amino acid sequence of a non-limiting example peptide that may be provided as R2 (Scheme 1 or 2) immediately preceding a C-terminal cysteine, wherein the penultimate amino acid of the peptide (from N-terminal to C-terminal of the polypeptide) prior to the photoamidation process (ie. Formula I in scheme 1 and 2) can be varied;
SEQ ID No: 2 represents the amino acid sequence of an example embodiment of the peptide that can form R2 (Scheme 1 or 2);
SEQ ID No: 3 represents the amino acid sequence of the peptide part (R2) of the starting material for producing glucagon-like peptide-1 (GLP-1) peptides;
SEQ ID No: 4 represents the amino acid sequence of the peptide part (R2) of the starting material for producing a pancreastatin (PST) inhibitor, more specifically pancreastatin inhibitor peptide-8 (PSTi8) peptide;
SEQ ID No: 5 represents the amino acid sequence of the peptide part (R2) of the starting material for producing pancreatic pYY (3-36) peptide;
SEQ ID No: 6 represents the amino acid sequence of the peptide part (R2) of the starting material for producing a luteinizing hormone-releasing hormone (LHRH) agonist;
SEQ ID No: 7 represents the amino acid sequence of the peptide part (R2) of the starting material for producing a gastrin releasing peptide (GRP) peptide;
SEQ ID No: 8 represents the amino acid sequence of the peptide part (R2) of the starting material for producing an adrenocorticotropic hormone (ACTH), more specifically Cosyntropin;
SEQ ID No: 9 represents the amino acid sequence of the peptide part (R2) of the starting material for producing a QRF-amide peptide;
SEQ ID No: 10 represents the amino acid sequence of the peptide part (R2) of the starting material for producing a GLP-1 receptor-neuropeptide Y receptor 2 co-agonist EP45;
SEQ ID No: 11 represents the amino acid sequence of the peptide part (R2) of the starting material for producing an entry inhibitor, more specifically Bulevirtide;
SEQ ID No: 12 represents the amino acid sequence of the peptide part (R2) of the starting material for producing osteocrin (OSTN) peptide;
SEQ ID No: 13 represents the amino acid sequence of the peptide part (R2) of the starting material for producing an antiretroviral drug, more specifically Enfuvirtide;
SEQ ID No: 14 represents the amino acid sequence of the peptide part (R2) of the starting material for producing a amylin receptor agonist, more specifically Pramlintide;
SEQ ID No: 15 represents the amino acid sequence of a non-limiting example peptide that may be provided as R2 (Scheme 1 or 2), for which a disulfide bond is desired therein;
SEQ ID No: 16 represents the amino acid sequence of the peptide part (R2) of the starting material for producing a GLP-1 receptor-amylin receptor co-agonist;
SEQ ID No: 17 represents the amino acid sequence of the peptide part (R2) of the starting material, consisting of the peptide for amidation and an N-terminal extension, for producing an a GLP-1 receptor-amylin receptor co-agonist; and
SEQ ID No: 18 represent the amino acid sequence of an example N-terminal extension which may form part the peptide part (R2).

### DRAWINGS

FIG. 1 depicts the overall reaction (Scheme 1) of the photochemical process for the manufacturing of a peptide or protein comprising a C-terminal α-amide; and

FIG. 2 depicts an embodiment of the reaction (Scheme 2) of the photochemical process for the manufacturing of a peptide or protein comprising a C-terminal α-amide, wherein chloro-7-nitrobenzofurazan is used as the photolabeling agent.

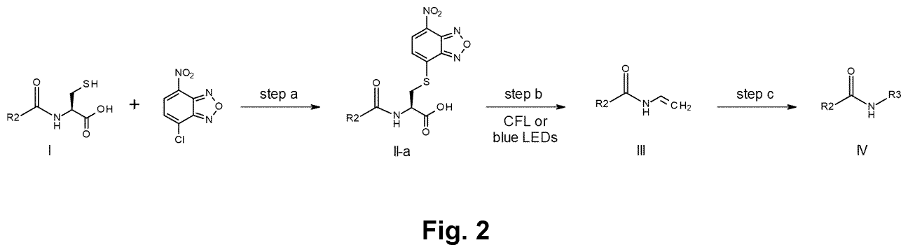

### DESCRIPTION

Structure-activity studies of therapeutic products such as the amylin peptide (Cooper et al., G. J. S. Molecular and functional characterization of amylin, a peptide associated with type 2 diabetes mellitus. Proc. Natl. Acad. Sci. USA 86, 9662-9666 (1989)), NPY (Rivier et al., Synthesis and hypertensive activity of neuropeptide Y fragments and analogues with modified N- or C-termini or D-substitutions. J. Med. Chem. 32, 597-601 (1989)) and others (Nuss et al., The current state of peptide drug discovery: back to the future? J. Med. Chem. 61, 1382-1414 (2018)) have revealed that C-terminal amidation is often required for full biological activity. However, the manufacturing-scale preparation of a peptide containing a C-terminal α-amide is particularly challenging. Thus, there is a need for a selective, low-cost and scalable semi-recombinant technology for the manufacturing of a peptide comprising a C-terminal amide.

The present invention relates to a photochemical process for the manufacturing of a peptide or protein comprising a C-terminal-amide. The instant method provides the advantage of being broad in scope, such that it may be utilised to produce a great variety of biologically active peptides with a C-terminal α-amide. As will become clear, the process for the manufacturing of a peptide or protein comprising a C-terminal α-amide disclosed herein is not bound to a particular peptide of protein (R2) and provides a method for the amidation of a broad range of peptides or proteins via a C-terminal cysteine, which may be referred to herein as a C-terminal cysteine amidation tag in view of its purpose in the photochemical process. The method is sufficiently specific to permit the C-terminal amidation of peptides and proteins even when it contains more than one cysteine therein (ie. cysteines within the peptide R2 in addition to the C-terminal cysteine amidation tag of use for manufacturing of the peptide or protein comprising a C-terminal α-amide). Also, or alternatively, another advantage of the invention is that the method is economically efficient due to the readily and commercially available starting materials and a simple reaction setup, which makes the process suitable for batch or in flow manufacturing, and permits the process to be of use in large scale manufacturing.

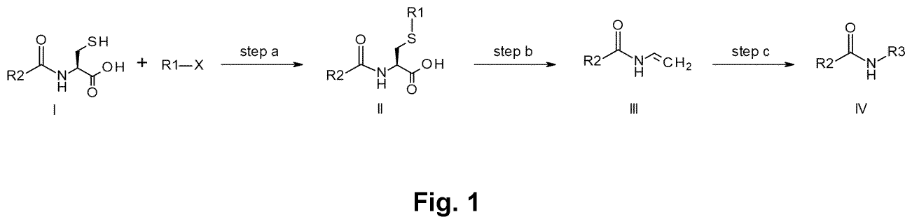

The invention is carried out as depicted in Scheme 1 (FIG. 1) by starting with a peptide or protein comprising a C-terminal cysteine residue (formula I) and reacting it with a photolabeling agent (R1-X) to obtain a peptide-photolabel conjugate (formula II), followed by exposing the peptide-photolabel conjugate to light to obtain a peptide or protein enamide (formula III), and subsequently converting the resulting enamide to the C-terminal α-amide (formula IV).

The photochemical amidation reaction as described above is split in three general reaction steps:
step (a): coupling of the peptide or protein comprising the C-terminal cysteine residue (formula I) with the photolabeling agent (R1-X) to obtain a peptide-photolabel conjugate (formula II);
Step (b): photochemical conversion of the resulting peptide-photolabel conjugate (formula II) to obtain a C-terminal enamide (formula III); and
Step (c): cleavage of the C-terminal enamide (formula III) to obtain the C-terminal a-amide (formula IV).

These steps (a), (b) and (c) are also depicted generally in Scheme 1 and within an example embodiment in Scheme 2. Each step may be characterised by its own reactants and conditions, as defined below.

It is understood that the process for the manufacturing of a peptide or protein comprising a C-terminal α-amide disclosed herein may be carried at varying strategic moments of the synthesis of biologically active peptides. In certain embodiments, the process for the manufacturing of a peptide or protein comprising a C-terminal α-amide disclosed herein can be used on intermediate products, including but not limited to precursors, prior to filtration steps such as high-performance liquid chromatography (HPLC) filtration for instance, prior to additional ligation steps and/or prior to cleavage of a peptide extension. The term extension here refers to at least one amino acid extending from the N-terminal of a desired peptide, such as may be the case for instance for a precursor. Such a peptide R2 may have the following structure:

### (Extension)-(Desired Peptide)

In such an embodiment, the combined extension and desired peptide form part of the peptide R2 during the photochemical process of the present disclosure, where the extension may be meant to be cleaved from peptide R2 in a subsequent step.

Proceeding with the photochemical amidation process early in the synthesis of a biologically active peptide may be considered advantageous-large-scale C-terminal amidation via the photochemical method described herein can take place well before the need for completion of process steps necessary to arrive to the final active pharmaceutical ingredient, minimizing losses of a downstream intermediate product that may be more costly in terms of materials having been used and/or manpower invested in the process of the synthesis.

The photochemical amidation reaction may also solve further problems that will be apparent from the disclosure of the exemplary embodiments.

In what follows, Greek letters may be represented by their symbol or the corresponding written name, for example: α=alpha; β=beta; ε=epsilon; γ=gamma; ω=omega; etc. Also, the Greek letter of μ may be represented by “u”, e.g. in μL=uL, or in μM=uM.

### General Reaction Steps

Step (a)—Coupling of Peptide or Protein Comprising C-Terminal Cysteine Residue (Formula I) with a Photolabeling Agent (R1-X) to Obtain a Peptide-Photolabel Conjugate (Formula II)

The first step (step (a)) of the photochemical amidation reaction may be referred to as the coupling of a peptide or protein comprising a C-terminal cysteine residue (also referred to as formula I) with a photolabel (R1) to obtain a peptide-photolabel conjugate (also referred to as formula II).

### C-Terminal Cysteine Residue (Formula I)

Formula I is a peptide or protein, comprising a polypeptide part (R2) and a C-terminal cysteine (Cys) residue. The polypeptide part R2 may also be referred to as a protein or peptide. The compound of formula I may be referred to as a peptide with a C-terminal cysteine (Cys) amidation tag. The compound of formula I may also be referred to as a polypeptide or a polypeptide comprising a C-terminal cysteine (Cys) residue. The C-terminal cysteine residue may be a residue of the amino acid cysteine, substituted at the C-terminus of the corresponding peptide or protein.

When referring to R2, it is to be understood as the polypeptide part of the peptide or protein of formula I, which does not include the C-terminal cysteine amidation tag and which will ultimately be provided with the C-terminal amide as per formula IV in accordance with the present method.

The photochemical reaction disclosed herein has been tested on peptides with varying penultimate amino acids (Example 3, Table 4, Entries 1-20). For this purpose, an example peptide R2 having a sequence IWTKDHEEVYEX (SEQ ID No. 1) with a varying penultimate amino acid (when R2 of SEQ ID No. 1 forms part of formula I of scheme 1, C-terminal is the cysteine amidation tag) was provided. In one embodiment, X is Ala. In one embodiment, X is Asp. In one embodiment, X is Glu. In one embodiment, X is Phe. In one embodiment, X is Gly. In one embodiment, X is His. In one embodiment, X is Ile. In one embodiment, X is Lys. In one embodiment, X is Leu. In one embodiment, X is Met. In one embodiment, X is Asn. In one embodiment, X is Pro. In one embodiment, X is Gln. In one embodiment, X is Arg. In one embodiment, X is Ser. In one embodiment, X is Thr. In one embodiment, X is Val. In one embodiment, X is Trp. In one embodiment, X is Tyr. For example, in the case where X of IWTKDHEEVYEX (SEQ. ID NO 1) is an Alanine (Ala), the resulting peptide R2 is IWTKDHEEVYEA (SEQ. ID NO 2), forming an peptide having the same amino acid sequence with an amide once having undergone the photochemical amidation reaction of the present application.

The photochemical amidation reaction has furthermore been used to prepare different intermediates of biologically active peptides (as depicted in Example 5, Table 5, Entries 1-12), more specifically the amidated precursors of the given biologically active peptides. In some embodiments, R2 may comprise an amino acid sequence as set out in any one of SEQ ID No. 3-14 and 16-17. In some embodiments, R2 may be an amido acid sequence as set out in any one of SEQ. ID NO.: 3, 4, 5, 6, 7, 8, 9, 10, 11, 12, 13, 14, 16 and 17. In some embodiments, R2 may comprise an amino acid sequence as set out in any one of SEQ. ID NO. 3, 4, 5, 16 and 17. In some embodiments, R2 may be an amino acid sequence as set out in any one of SEQ. ID NO. 3, 4, 5, 16 and 17. In some embodiments, R2 may comprise an amino acid sequence as set out in SEQ ID No. 16.

In some embodiments, the photochemical amidation reaction may be used to prepare glucagon-like peptide-1 (GLP-1) peptides or precursors thereof. The photochemical amidation reaction may be used to prepare amylin receptor agonists such as Pramlintide (the active pharmaceutical ingredient in Symlin™), or precursor thereof. The photochemical amidation reaction may be used to prepare glucose-dependent insulinotropic polypeptide (GIP) peptides or precursors thereof. The photochemical amidation reaction may be used to prepare GIP and GLP-1 receptor co-agonists such as Tirzepatide, or precursors thereof. The photochemical amidation reaction may be used to prepare corticotropin-releasing factor (CFR) peptides such as urocortin-2 (UCN2) peptide, or precursors thereof. The photochemical amidation reaction may be used to prepare pancreatic pYY (3-36) peptide or precursors thereof. The photochemical amidation reaction may be used to prepare pancreastatin (PST) inhibitors, such as pancreastatin inhibitor peptide-8 (PSTi8) peptide, or precursors thereof. The photochemical amidation reaction may be used to prepare luteinizing hormone-releasing hormone (LHRH) agonist or precursors thereof. The photochemical amidation reaction may be used to prepare gastrin releasing peptide (GRP) peptides or precursors thereof. The photochemical amidation reaction may be used to prepare adrenocorticotropic hormones (ACTH) such as Cosyntropin (active pharmaceutical ingredient of Cortrosyn™), or precursors thereof. The photochemical amidation reaction may be used to prepare QRF-amide peptides or precursors thereof. The photochemical amidation reaction may be used to prepare GLP-1 receptor-neuropeptide Y receptor 2 co-agonist, such as EP45, or precursors thereof. The photochemical amidation reaction may be used to prepare entry inhibitors, such as Bulevirtide (active pharmaceutical ingredient of Hepcludex™), or precursors thereof. The photochemical amidation reaction may be used to prepare natriuretic peptides such as osteocrin (OSTN) peptide or precursors thereof. The photochemical amidation reaction may be used to prepare antiretroviral drugs such as Enfuvirtide or precursors thereof. The photochemical amidation reaction may be used to prepare GLP-1 receptor-amylin receptor co-agonists or precursors thereof, such as that represented by SEQ ID NO. 16 or 17.

The photochemical amidation reaction may be used to prepare glucagon-like peptide-1 (GLP-1) peptides, amylin receptor agonists such as Pramlintide (the active pharmaceutical ingredient in Symlin™), glucose-dependent insulinotropic polypeptide (GIP) peptides, GLP-1 receptor co-agonists such as Tirzepatide, corticotropin-releasing factor (CFR) peptides such as Urocortin-2 (UCN2) peptide, pancreatic pYY (3-36) peptide, pancreastatin (PST) inhibitors such as pancreastatin inhibitor peptide-8 (PSTi8) peptide, luteinizing hormone-releasing hormone (LHRH) agonist, Gastrin releasing peptide (GRP) peptides, adrenocorticotropic hormones (ACTH) such as Cosyntropin (active pharmaceutical ingredient of Cortrosyn™), QRF-amide peptides, GLP-1 receptor-neuropeptide Y receptor 2 co-agonist such as EP45, entry inhibitors such as Bulevirtide (active pharmaceutical ingredient of Hepcludex™), a natriuretic peptide such as osteocrin (OSTN) peptide, antiretroviral drugs such as Enfuvirtide, and GLP-1 receptor-amylin receptor co-agonists such as those comprising the amino acid sequence of SEQ ID No. 16, or precursors thereof.

The photochemical amidation reaction may be used to prepare glucagon-like peptide-1 (GLP-1) peptides, amylin receptor agonists such as Pramlintide (the active pharmaceutical ingredient in Symlin™), pancreatic pYY (3-36) peptide, pancreastatin (PST) inhibitors such as pancreastatin inhibitor peptide-8 (PSTi8) peptide, luteinizing hormone-releasing hormone (LHRH) agonists and antagonists, Gastrin releasing peptide (GRP) peptides, adrenocorticotropic hormones (ACTH) such as Cosyntropin (active pharmaceutical ingredient of Cortrosyn™), QRF-amide peptides, GLP-1 receptor-neuropeptide Y receptor 2 co-agonist such as EP45, entry inhibitors, such as Bulevirtide (active pharmaceutical ingredient of Hepcludex™), Osteocrin, antiretroviral drugs such as Enfuvirtide, and GLP-1 receptor-amylin receptor co-agonists such as those comprising the amino acid sequence of SEQ ID No. 16, or precursors thereof.

The photochemical amidation reaction may be used to prepare glucagon-like peptide-1 (GLP-1) peptides, pancreatic pYY (3-36) peptide, pancreastatin (PST) inhibitors such as pancreastatin inhibitor peptide-8 (PSTi8) peptide, GLP-1 receptor-amylin receptor co-agonists such as those comprising the amino acid sequence of SEQ ID No. 16, or precursors thereof.

The photochemical amidation reaction may be used to prepare glucagon-like peptide-1 (GLP-1) peptides, pancreatic pYY (3-36) peptide, and GLP-1 receptor-amylin receptor co-agonists such as those comprising the amino acid sequence of SEQ ID No. 16, or precursors thereof.

The preparation of GLP-1 peptides as disclosed herein includes native human GLP-1 (7-36)-amide (such as starting peptide R2 may be GLP-1 (7-36), SEQ ID No. 3) as well as analogues thereof (GLP-1 analogues)). The photochemical amidation reaction may be used to prepare GLP-1 (7-36)-amide. The photochemical amidation reaction may be used to prepare a GLP-1 receptor-amylin receptor co-agonists. The preparation of GLP-1 receptor-amylin receptor co-agonists disclosed herein includes the preparation of co-agonists comprising an amino acid sequence as set out in SEQ ID No. 16.

In some embodiments of the photochemical amidation reaction, R2 may comprise a sequence corresponding to a precursor of a bioactive peptide product, such as a GLP-1 receptor-amylin receptor co-agonist or amylin receptor agonists. The photochemical amidation reaction may be used to prepare a GLP-1 receptor-amylin receptor co-agonist precursor comprising an amino acid sequence as set out in SEQ ID No. 16. The photochemical amidation reaction may be used to prepare a GLP-1 receptor-amylin receptor co-agonist precursor consisting of an amino acid sequence as set out in SEQ ID No. 16. The photochemical amidation reaction may be used to prepare a GLP-1 receptor-amylin receptor co-agonist precursor consisting of an amino acid sequence as set out in SEQ ID No. 17.

In some embodiments of the photochemical amidation reaction, R2 may be a peptide with an N-terminal extension. The extension may be any combination of amino acids. The extension may be between 1-60 amino acids. The extension may be between 1-50 amino acids. The extension may be between 1-40 amino acids. The extension may be between 1-20 amino acids. The extension may be between 1-15 amino acids. The extension may be between 5-15 amino acids. The extension may be 14 amino acids.

In some embodiments, the photochemical amidation reaction may be used to prepare a GLP-1 receptor-amylin receptor co-agonist precursor with an N-terminus extension. In some embodiments, R2 may be a peptide with an amino acid sequence as set out in SEQ ID No. 16 plus any type of N-terminal extension. R2 may be a peptide with an N-terminal extension with an amino acid sequence as set out in SEQ ID No. 18. R2 may be a peptide with an amino acid sequence as set out in SEQ ID No. 16 plus an N-terminal extension with an amino acid sequence as set out in SEQ ID No. 18. R2 may be a peptide with an amino acid sequence as set out in SEQ ID No. 17.

In some embodiments of the photochemical amidation reaction, R2 may comprise an amino acid sequence as set out in any one of any one of SEQ ID Nos. 3-14, 16-17. R2 may comprise an amino acid sequence as set out in any one of SEQ ID Nos. 3-14, 16 plus an N-terminal extension. R2 may be an amino acid sequence as set out in any one of SEQ ID Nos: 3-14, 16-17. R2 may be an amino acid sequence as set out in any one of SEQ ID NOs: 3-14, 16 plus an N-terminal extension.

In some embodiments of the photochemical amidation reaction, the peptide or protein comprising a C-terminal cysteine residue (formula I) may be reacted with a photolabeling agent (R1-X). In another embodiment, a compound of formula I may be coupled to a photolabel (R1).

### Photolabeling Agent (R1-X)

A photolabeling agent (herein also identified as R1-X) may be defined as a chemical compound that contains a photo-excitable moiety and that is capable of coupling to a peptide. The photolabeling agent (R1-X) may be defined as a compound capable of undergoing a photochemical reaction.

The photolabeling agent (R1-X) comprises a photolabel defined herein as R1 and a leaving group defined herein as X. Herein, the photolabel (R1) may be coupled to a peptide or protein comprising a C-terminal cysteine (formula I) by reacting the peptide or protein with the photolabeling agent (R1-X) under the conditions described herein.

In one embodiment of the photochemical amidation reaction, the photolabeling agent (R1-X) may be selected from the group consisting of 3-bromo-1H-pyrrole-2,5-dione, 4-chloro-7-nitrobenzofurazan, 2-bromo-1,4-naphthoquinone, 1-fluoro-2,4-dinitrobenzene and 4-fluoro-7-sulfamoylbenzofurazan.

3-Bromo-1H-pyrrole-2,5-dione may also be referred to as 2-bromomaleimide and may be defined as Chem 1:

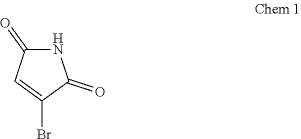

4-chloro-7-nitrobenzofurazan may also be referred to as NBD-CI and may be defined as Chem 2:

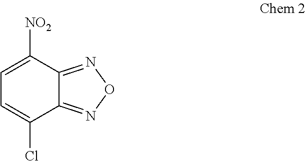 [SMILES: O=[N+]([O-])c1ccc(Cl)c2nonc12]

In some embodiments, the photolabeling agent (R1-X) may be 3-bromo-1H-pyrrole-2,5-dione (Chem. 1) or 4-chloro-7-nitrobenzofurazan (Chem. 2). The photolabeling agent (R1-X) may be 3-bromo-1H-pyrrole-2,5-dione (Chem. 1). The photolabeling agent (R1-X) may be 4-chloro-7-nitrobenzofurazan (Chem. 2).

In some embodiments, between 1 and 5 equivalents of the photolabeling agent (R1-X) relative to the peptide (formula I) may be provided. In some embodiments, between 1 and 3 equivalents of the photolabeling agent (R1-X) relative to the peptide (formula I) may be provided. In some embodiments, between 1.5 and 2.5 equivalents of the photolabeling agent (R1-X) relative to the peptide (formula I) may be provided. In some embodiments, between 1.8 and 2.2 equivalents of the photolabeling agent (R1-X) relative to the peptide (formula I) may be provided. In some embodiments, about 2 equivalents of the photolabeling agent (R1-X) relative to the peptide (formula I) may be provided.

Reaction Conditions of Peptide or Protein Comprising C-Terminal Cysteine Residue with Photolabeling Agent
Step (a) of the photochemical amidation reaction may take place in an aqueous reaction buffer. The photochemical amidation reaction may take place in an aqueous reaction buffer, such as an aqueous buffer selected from the group consisting of bis-tris methane (may be referred to as bis-tris), tris, triethanolamine and phosphate. Steps (a) of the photochemical amidation reaction may take place in an aqueous buffer at pH 4-9. Steps (a) of the photochemical amidation reaction may take place in an aqueous buffer comprising bis-tris methane at pH 4-9. Steps (a) of the photochemical amidation reaction may take place in an aqueous buffer at pH 6-8. Steps (a) of the photochemical amidation reaction may take place in an aqueous buffer comprising bis-tris methane at pH 6-8. Steps (a) of the photochemical amidation reaction may take place in an aqueous buffer at pH 6-7. Step (a) of the photochemical amidation reaction may take place in an aqueous reaction buffer comprising bis-tris methane at pH 6-7. Step (a) of the photochemical amidation reaction may take place in an aqueous reaction buffer at pH 6.3-7. Step (a) of the photochemical amidation reaction may take place in an aqueous reaction buffer comprising bis-tris methane at about pH 6.3-7. Step (a) of the photochemical amidation reaction may take place in an aqueous reaction buffer at about pH 6.4. Step (a) of the photochemical amidation reaction may take place in an aqueous reaction buffer comprising bis-tris methane at about pH 6.4. Step (a) of the photochemical amidation reaction may take place in an aqueous reaction buffer comprising bis-tris methane at about pH 6.3. Step (a) of the photochemical amidation reaction may take place in an aqueous reaction buffer comprising bis-tris methane at about pH 7. Step (a) of the photochemical amidation reaction may take place in an aqueous reaction buffer comprising about 25 mM bis-tris methane at about pH 6.4. Step (a) of the photochemical amidation reaction may take place in an aqueous reaction buffer comprising 25 mM bis-tris methane at about pH 6.3.
Step (a) of the photochemical amidation reaction may take place in an aqueous reaction buffer with glycine. Step (a) of the photochemical amidation reaction may take place in an aqueous reaction buffer with about 50 mM of glycine.

In another embodiment, step (a) of the photochemical amidation reaction may take place in an aqueous reaction buffer comprising about 25 mM bis-tris methane at about pH 6.4 with about 50 mM glycine. Step (a) of the photochemical amidation reaction may take place in an aqueous reaction buffer comprising about 25 mM bis-tris methane at about pH 6.3 with about 50 mM glycine.

In some embodiments, step (a) of the photochemical amidation reaction may take place at room temperature, which defined herein as being between 15-25° C. Step (a) of the photochemical amidation reaction may take place at a temperature between 4-80° C. Step (a) of the photochemical amidation reaction may take place at a temperature between 10-60° C. Step (a) of the photochemical amidation reaction may take place at a temperature between 10-40° C. Step (a) of the photochemical amidation reaction may take place at a temperature between 10-30° C. Step (a) of the photochemical amidation reaction may take place at a temperature between 15-30° C. Step (a) of the photochemical amidation reaction may take place at a temperature of about 20° C.

A surfactant may be added to the reaction buffer prior to photochemical conversion (described in step (b) below). This surfactant may be used to improve the solubility of the photolabel intermediates. For example, and as will be discussed below regarding the reaction with disulfide containing peptides, the surfactants may provide improved solubility of the 4-chloro-7-nitrobenzofurazan (NBD-CI, Chem. 2) alkylated intermediates when the NBD-CI photolabeling agent is used to globally alkylate all the cysteines within a peptide (R2).

In some embodiments, a surfactant selected from the group consisting of sodium dodecyl sulfate (SDS), sodium desoxycholate (SDC), 3-[(3-cholamidopropyl) dimethylammonio]-1-propanesulfonate (CHAPS), octyl phenol ethoxylate (Triton™ X-100, ThermoFisher Scientific Cat. No. 28314, 85111, 85112), polysorbate 20 (Tween-20, ThermoFisher Scientific Cat. No. 28320, 85113, 85115), Tetrapropylammonium hydroxide (TAPH-40) and sodium octanoate may be added to the reaction buffer. In some embodiments, sodium dodecyl sulfate (SDS) may be added to the reaction buffer. In some embodiments, sodium desoxycholate (SDC) may be added to the reaction buffer. In some embodiments, 3-[(3-cholamidopropyl) dimethylammonio]-1-propanesulfonate (CHAPS) may be added to the reaction buffer. In some embodiments, sodium octanoate may be added to the reaction buffer. In some embodiments, octyl phenol ethoxylate (Triton™ X-100, ThermoFisher Scientific Cat. No. 28314, 85111, 85112) may be added to the reaction buffer. In some embodiments, polysorbate 20 (Tween-20, ThermoFisher Scientific Cat. No. 28320, 85113, 85115) may be added to the reaction buffer. In some embodiments, Tetrapropylammonium hydroxide (TAPH-40) may be added to the reaction buffer.

The surfactant may be added to the reaction buffer in a concentration between 40-80 mM. The surfactant may be added to the reaction buffer in a concentration between 50-70 mM. The surfactant may be added to the reaction buffer in a concentration between 55-65 mM. The surfactant may be added to the reaction buffer in a concentration of about 60 mM. In some embodiments, no surfactant may be added to the reaction buffer.

Step (b)—Photochemical Conversion of the Resulting Peptide-Photolabel Conjugate (Formula II) to Obtain a C-Terminal Enamide (Formula III)

The second step (step (b)) of the reaction may be referred to as the photochemical conversion of conjugate (also referred to as formula II) to C-terminal enamide (also referred to as formula III). Step (b) may also be referred to as the conversion of the peptide-photolabel conjugate to obtain the C-terminal enamide. In some embodiments, the peptide-photolabel conjugate may be referred to as a conjugate.

The photochemical conversion of step (b) may be obtained by subjecting the peptide-photolabel conjugate of formula II to light. Written otherwise, the photochemical conversion of step (b) may be obtained by irradiating the peptide-photolabel conjugate. The light shined on the conjugate of formula II may be absorbed by the photolabel, leading to a light promoted chemical reaction, which may be referred to herein generally as a photochemical conversion, to ultimately form the C-terminal enamide of formula III. The photochemical conversion of step (b) may be referred to as a photolytic cleavage by radical decarboxylation of the C-terminal. The enamide of formula III may be said to be monosubstituted.

It is understood that the effective wavelength for this photochemical conversion may vary based on the photolabel R1 coupled to the C-terminal cysteine residue (formula I) in part a. Further it is understood that a variety of light sources may be used to generate the light spectrum desired for the photochemical reaction to occur. The light sources may be selected from fluorescent lamps, light-emitting diodes, mercury lamps, lasers, etc. In some embodiments, the light source may be ambient light, such as light provided by the sun. In some embodiments, the light source may be a compact fluorescent lamp (CFL). The light source may be a light emitting diode (LED). The LED may be a monochromatic LED. In some embodiments, more than one light source may be used for irradiating the conjugate of formula II to light uniformly. The light source may be more than one CLF. The light source may be more than one LED. The light source may be a series of LEDs uniformly arranged to irradiate the conjugate. The light source may be arrays of LEDs, which can be referred to as strips of LEDs when arranged in a row.

The light provided may be white light. The wavelength of the light provided may be within the visible light spectrum. The wavelength of the light provided may be between 300-700 nm. The wavelength of the light provided may be between 365-550 nm. The wavelength of the light provided may be between 365-500 nm. The wavelength of the light provided may be between 365-450 nm. The wavelength of the light provided may be about 365 nm. The wavelength of the light provided may be between 400-500 nm. The wavelength of light provided may be between 400-470 nm. The wavelength of light provided may be between 400-450 nm. The wavelength of light provided may be between 405-430 nm. The wavelength provided may be of 365 nm. The wavelength provided may be of 405 nm. The wavelength provided may be of 430 nm. The wavelength provided may be of 450 nm. The source of light may be LEDs providing light having a wavelength of 365 nm. The source of light may be a CFL providing white light. The source of light may be LEDs providing light having a wavelength of 405 nm. The source of light may be LEDs providing light having a wavelength of 430 nm. The source of light may be LEDs providing light having a wavelength of 450 nm. The source of light may be an array of monochromatic LEDs providing light having a wavelength of 365 nm. The source of light may be an array of monochromatic LEDs providing light having a wavelength of 405 nm. The source of light may be an array of monochromatic LEDs providing light having a wavelength of 430 nm. The source of light may be an array of monochromatic LEDs providing light having a wavelength of 450 nm.

It is understood that when a single wavelength is referred to herein, reference is made to a spectrum of light having a spectral width generally centred at said wavelength. For instance, should the light provided by the light source be at a wavelength of 405 nm, it is understood that it is not to be interpreted as strictly limited to the light of 405 nm wavelength, but be directed towards a spectrum with a given width encompassing the 405 nm wavelength. In the case of light having a wavelength of 405 nm, and for a full width at half maximum (FWHM) of 50 nm for instance, one would understand that the effective wavelength irradiating the conjugate extends between 380-430 nm.

The light source may be combined with spectral filters, such as high-pass filters, low-pass filters and/or bandgap filters, which may be considered to form part of the light source and may further restrict the spectral range irradiating the conjugate.

In some embodiments, the spectral width of the light may have a FWHM of 50 nm. The spectral width of the light may have a FWHM of 40 nm. The spectral width of the light may have a FWHM of 30 nm. The spectral width of the light may have a FWHM of 20 nm. The spectral width of the light may have a FWHM of 10 nm.

The reaction condition for photochemical conversion of the peptide-photolabel conjugate (formula II) to obtain a C-terminal enamide (formula III) may correspond to that provided for the coupling of peptide or protein comprising C-terminal cysteine residue (formula I) with a photolabeling agent (R1-X) to obtain a peptide-photolabel conjugate (formula II) in step (a) above. For instance, step (b) may take place in the same aqueous reaction buffer for step (a). Likewise, the step (b) may take place at a temperature corresponding to that of step (a).

Step (c)—Cleavage of the C-Terminal Enamide (Formula III) to Obtain the C-Terminal α-Amide (Formula IV)

The third and last step of the reaction (step (c)) may be referred to as the cleavage of the resulting C-terminal enamide (also referred to as formula III) to obtain the C-terminal a-amide (also referred to as formula IV). In some embodiments, the cleavage may be referred to as the conversion of the C-terminal enamide. The C-terminal enamide may be referred to as enamide. The C-terminal-amide may be referred to as a C-terminal amide.

The cleavage of the enamide to a C-terminal α-amide may be carried out using different cleavage methods. The cleavage may be carried out using acidolysis. In some embodiments, the cleavage may be carried out using acidolysis with an acid that may be categorized as being a strong acid, where strong acid here is defined as an acid with a pKa below or equal to 4. In some embodiments, the cleavage reagent may have a pKa below or equal to 4. In some embodiments, the cleavage reagent may have a pKa below or equal to 3. In some embodiments, the cleavage may be carried out using acidolysis with a weak acid (ie. an acid with a pKa above 4) with the addition of Lewis acids to accelerate the acidolysis.

The cleavage may be carried out using inverse-electron demand Diels-Alder (IEDDA). In some embodiments, the enamide may be cleaved by acid mediated hydrolysis. The enamide may be hydrogenated. The enamide may be cleaved by acid catalysed, oxidizing agent mediated hydrolysis or by an Inverse Electron Demand Diels-Alder reaction. The enamide may be cleaved by acid catalysed or by oxidizing agent mediated hydrolysis. The enamide may be cleaved by acid catalysed hydrolysis. The enamide may be cleaved by oxidizing agent mediated hydrolysis. The enamide may be cleaved by an Inverse Electron Demand Diels-Alder reaction.

In some embodiments, the vinyl amide may be reduced to N-ethyl amide. In some embodiments, the vinyl amide may be used for conjugation, such as a thiol-ene reaction.

In some embodiments, an additive may be added to the step. The additives may be selected from, but not limited to, the group consisting of methionine, indole or caffeic acid.

### Reaction Conditions of Enamide Cleavage

The different cleavage methods, as set out above, may require different reaction conditions, such as different cleavage reagents and temperature. In some embodiments, the cleavage reagent is added to the reaction with the intermediate of formula III therein.

One understands that some variables of the reaction condition may remain constant throughout steps (a), (b) and (c). For instance, in some embodiments, the photochemical amidation reaction takes place at a constant temperature. In other embodiments, the temperature may be change between the steps (a), (b) and/or (c). In other embodiments, the conditions of the photochemical reaction at steps (a), (b) and/or (c) may be different to one another.

In some embodiments, the cleavage reagent may be selected from the group consisting of trifluoroacetic acid (TFA), hydrochloric acid (HCl), Sulfuric acid (H₂SO₄), tosylic acid (TsOH), phosphoric acid (H₃PO₄), oxalic acid, 3,6-diphenyl-1,2,4,5-tetrazine, and 6,6′-(1,2,4,5-tetrazine-3,6-diyl) dinicotinic acid. The cleavage reagent may be selected from the group consisting of trifluoroacetic acid (TFA), hydrochloric acid (HCl), Sulfuric acid (H₂SO₄), tosylic acid (TsOH), phosphoric acid (H₃PO₄), oxalic acid and 6,6′-(1,2,4,5-tetrazine-3,6-diyl) dinicotinic acid. The cleavage reagent may be selected from the group consisting of trifluoroacetic acid (TFA), hydrochloric acid (HCl), Sulfuric acid (H₂SO₄), tosylic acid (TsOH) and 6,6′-(1,2,4,5-tetrazine-3,6-diyl) dinicotinic acid. The cleavage reagent may be trifluoroacetic acid (TFA). The cleavage reagent may be hydrochloric acid (HCl). The cleavage reagent may be sulfuric acid (H₂SO₄). The cleavage reagent may be phosphoric acid (H₃PO₄). The cleavage reagent may be tosylic acid (TsOH). The cleavage reagent may be 6,6′-(1,2,4,5-tetrazine-3,6-diyl) dinicotinic acid.

In some embodiments, the cleavage reagent may be between 2-8% v/v trifluoroacetic acid (TFA). The cleavage reagent may be between 4-6% v/v trifluoroacetic acid (TFA). The cleavage reagent may be 5% v/v trifluoroacetic acid (TFA).

In some embodiments, the cleavage reagent may be 0.5-2 M phosphoric acid (H₃PO₄). The cleavage reagent may be 0.5-1.5 M phosphoric acid (H₃PO₄). The cleavage reagent may be 0.7-1.3 M phosphoric acid (H₃PO₄). The cleavage reagent may be 0.8-1.2 M phosphoric acid (H₃PO₄). The cleavage reagent may be about 1 M phosphoric acid (H₃PO₄).

In some embodiments, the enamide cleavage may be performed at a temperature between 0 and 80° C. The enamide cleavage may be performed at a temperature between 2° and 60° C. The enamide cleavage may be performed at a temperature between 2° and 40° C. The enamide cleavage may be performed at a temperature between 3° and 40° C. The enamide cleavage may be performed at a temperature of 37° C. The enamide cleavage may be performed at a temperature between 2° and 26° C. The enamide cleavage may be performed at a temperature between 2° and 23° C. The enamide cleavage may be performed at a temperature of about 20° C. The enamide cleavage may be performed at a temperature of about 23° C. The enamide cleavage may be performed at room temperature, which may also be referred to as ambient temperature.

The reaction conditions may vary depending on the procedure of which the reaction is carried out.

### Batch Procedure

The photochemical amidation reaction described herein may be performed in a batch procedure. The batch procedure may also be referred to as batch scale reaction. In some embodiments, the photochemical amidation process may be performed in in the same reaction vessel, which may be referred to as a “single pot” reaction. It is to be understood that, while a reaction may take place in a single reaction vessel, it should not be interpreted to state that the reactions conditions therein are to remain constant. For instance, steps (a) and (b) of the photochemical amidation reaction described herein may take place at room temperature in a reaction vessel, while the temperature may be adjusted to, for instance about 37° C., for step (c) while nevertheless taking place in the same reaction vessel.

In other embodiments, steps (a), (b) and/or (c) may be performed in different reaction vessels. The reaction conditions may vary depending on the photolabeling agent (R1-X) and the cleavage reagents used, as described above.

### Photo-Flow Procedure

The photochemical amidation reaction may be performed in a photo-flow procedure, wherein the solution is pumped through a circuit while being irradiated by a light source. The reaction may be performed in a flow reactor providing these conditions. The flow reactor may be referred to as a photo-flow reactor, photochemical reactor or more generally as a reactor.

In some embodiments, the reaction performed in a flow reactor may also be referred to as photo-flow process or reaction. In some embodiments, the reaction performed in a flow reactor may also be referred to as a photo-flow procedure.

The flow reactor may operate at a temperature between 10 to 100° C. The flow reactor may operate at a temperature between 20 to 60° C. The flow reactor may operate at a temperature between 20 to 40° C. The flow reactor may operate at a temperature between 20 to 30° C. The flow reactor may operate at a temperature between 25 to 30° C. The flow reactor may operate at a temperature of about 26° C.

The photochemical amidation reaction may be performed at different flow rates. The flow rate here refers to the volumetric flow rate which may be defined as the volume of fluid which passes through the system per unit of time.

The flow rate may be between 0.200 and 50.00 mL/min. The flow rate may be between 1.00 and 25.00 mL/min. The flow rate may be 5.00 and 10.00 mL/min. The flow rate may be between 6.00 and 9.00 mL/min. The flow rate may be about 8.00 mL/min. The flow rate may be about 10 mL/min. The flow rate may be about 20 mL/min.

The flow reactor volume may be between 1 to 10 mL. The flow reactor volume may be between 2 and 5 mL. The flow reactor volume may be about 2 mL. The flow reactor volume may be about 2.7 mL. The flow reactor volume here may be regarded as the volume that may be subject to irradiation at any given time by the light source of the flow reactor.

The reaction may also be carried out using different residence times. The residence time here may be defined as a measure of how long a fluid stays in the flow reactor. Written otherwise, it is a measurement of time between the moment the solution enters the flow reactor, forming part of the flow reactor volume and capable of being irradiated by the flow reactor, and the moment the solution exits the flow reactor, no longer forming part of the flow reactor volume and no longer being irradiated by the flow reactor. It may also be given by the ratio of flow reactor volume to the overall flow rate.

The residence time in the flow reactor may be between 0.04 and 50 min. The residence time in the flow reactor may be between 0.1 and 10 min. The residence time in the flow reactor may be between 0.1 and 1 min. The residence time in the flow reactor may be between 0.1 and 0.5 min. The residence time in the flow reactor may be about 0.25 min.

The flow reactor may be a Vapourtec UV-150 photochemical reactor. The flow reactor may be a Corning® Lab Photo Reactor. It will be understood that any equivalent photochemical reactor which permits the irradiation of a circulating solution for the purposes of photochemical conversion as described in step (b) may be used without departing from the present disclosure.

In some embodiments, the flow reactor light source may irradiate at a wavelength between 300 nm and 700 nm. The flow reactor light source may be a series of LEDs irradiating at a wavelength between 365 and 525 nm. The flow reactor light source may be a series of monochromatic LEDs irradiating at a wavelength between 400 and 450 nm. The flow reactor light source may be a series of monochromatic LEDs irradiating at a wavelength of 430 nm. The flow reactor light source may be a series of monochromatic LEDs irradiating at a wavelength between 390 and 420 nm. The flow reactor light source may be a series of monochromatic LEDs irradiating at a wavelength of about 405 nm.

In some embodiments, the flow reactor light source may irradiate at a radiant power between 3 and 150 watts. The flow reactor light source may irradiate at a radiant power between 5 and 100 watts. The flow reactor light source may irradiate at a radiant power between 10 and 80 watts. The reactor light source may irradiate at a radiant power between 9 and 24 watts.

### Batch—Photo-Flow Hybrid Procedure

The photochemical amidation reaction may be performed in a batch-photo-flow hybrid procedure, in which only a portion of the photochemical amidation reaction may be completed via a photo-flow procedure. Written otherwise, in some embodiments, only some of the steps of the reaction occur in a flow reactor, where the remaining steps may occur in a batch procedure, in a reaction vessel for instance. When only a single step or only some steps of the photochemical amidation reaction occurs in a photo-flow reactor, the reaction may generally be referred to being completed in a hybrid procedure.

In some embodiments, the photochemical conversion of the peptide-photolabel conjugate to obtain the C-terminal enamide of the reaction may be performed in a flow reactor. In some embodiments, the coupling of the peptide comprising a C-terminal cysteine residue with a photolabel to obtain a peptide-photolabel conjugate and the photochemical conversion of the peptide-photolabel conjugate to obtain the C-terminal enamide of the reaction may be performed in a flow reactor. In some embodiments, the photochemical conversion of the peptide-photolabel conjugate to obtain the C-terminal enamide and the cleavage of the C-terminal enamide to obtain the C-terminal α-amide of the reaction may be performed in a flow reactor. In some embodiments, only the photochemical conversion of the peptide-photolabel conjugate to obtain the C-terminal enamide of the reaction may be performed in a flow reactor. In some embodiments, the photochemical amidation reaction steps which are not performed in a flow reactor may be performed performed via batch procedures. In some embodiments, the photochemical amidation reaction steps which are not performed in a flow reactor may be performed performed via batch procedures in reaction vessels.

### Photochemical Reaction of Disulfide Bond Containing Peptides

The photochemical amidation reaction of the present application may take place with peptides R2 that contain cysteines residues therein. In other words, formula I of Scheme 1 or 2 contains a peptide which comprises at least one cysteine (Cys) residue in addition to the C-terminal cysteine (Cys) residue for the photochemical amidation reaction. The cysteines within the peptide R2 may form disulfide bonds.

In such an embodiment, the photochemical amidation process may proceed as generally described above while further being subject to the below.

### Global Coupling of Photolabling Agent

The photolabeling agent (R1-X) may couple to the C-terminal cysteine residue, as described in step (a) above, as well as to the additional cysteine residues present in the peptide R2. This may be referred to as global coupling of the cysteines. The cysteines forming part of the peptide R2 having been coupled with the photolabel R1 may generally be referred to as photolabel-protected cysteines.

The photolabeling agent may be added in a manner proportional to the quantity of cysteines to be coupled with the photolabeling agent. The equivalents of photolabeling agent may increase proportional to the number of disulfide bonds to be formed within the amidated peptide.

In some embodiments, between 0.5 and 3 additional equivalents of the photolabeling agent (R1-X) relative to the peptide (formula I) may be provided for each disulfide bond to be formed in the peptide (R2). Between 1 and 2.5 additional equivalents of the photolabeling agent (R1-X) relative to the peptide (formula I) may be provided for each disulfide bond to be formed in the peptide (R2). Between 1.5 and 2.5 additional equivalents of the photolabeling agent (R1-X) relative to the peptide (formula I) may be provided for each disulfide bond to be formed in the peptide (R2). In some embodiments, about 2 additional equivalents of the photolabeling agent (R1-X) relative to the peptide (formula I) may be provided for each disulfide bond to be formed in the peptide (R2). The term additional here refers to photolabeling agent equivalents which are to be summed to the photolabeling agent equivalents one would provide in the general reaction at step (a) should the peptide R2 have no disulfide bonds to be formed.

In some embodiments, between 3 and 5 total equivalents of the photolabeling agent (R1-X) relative to the peptide (formula I) may be provided for step a. Between 3.5 and 4.5 total equivalents of the photolabeling agent (R1-X) relative to the peptide (formula I) may be provided for step a. Between 3.8 and 4.2 total equivalents of the photolabeling agent (R1-X) relative to the peptide (formula I) may be provided for step a. In some embodiments, about 4 total equivalents of the photolabeling agent (R1-X) relative to the peptide (formula I) may be provided for step a. The term total refers to the resulting amount of photolabeling equivalents provided in the reaction during step (a) of the photochemical amidation reaction.

In some embodiments, about 4 total equivalents of 4-chloro-7-nitrobenzofurazan (NBD-CI) relative to the peptide may be provided for step a.

### Selective C-Terminal Photochemical Conversion

The peptide-photolabel conjugate may be irradiated in the manner generally disclosed in step b. The photolabel found at the C-terminal of the conjugate may go through the photochemical conversion such as to obtain the C-terminal enamide, while the remaining cysteine coupled photolabels may remain unaffected.

In some embodiments, the photochemical conversion step (b) disclosed herein may be said to be selective to the C-terminal of the peptide-photolabel conjugate. In some embodiments, the photochemical conversion step (b) disclosed herein may be said to be selective to the C-terminal cysteine of the peptide coupled with the photolabel.

### Releasing & Oxidation

The cleavage of the C-terminal enamide to obtain the C-terminal α-amide may be carried out as described in step (c).

Further, the release of the photolabel in the photolabel-protected cysteines of the peptide R2 may be carried out. The release of the photolabel-protected cysteines in the peptide R2 may be carried out before the cleavage of the C-terminal enamide to obtain the C-terminal α-amide of step (c). The release of the photolabel-protected cysteines in the peptide R2 may be carried out simultaneously to the cleavage of the C-terminal enamide to obtain the C-terminal α-amide of step (c).

A nucleophilic sulfide may be provided, leading to the release of the photolabels from photolabel-protected peptide cysteines. In some embodiments, a nucleophilic sulfide selected from the group consisting of cysteamine, cysteine, dithiothreitol (DTT), 3,6-dioxa-1,8-octanedithiol (DODT), 2-mercaptoethanol, glutathione (GSH) and acetylcysteine may be provided. In some embodiments, cysteamine may be provided as a nucleophilic sulfide.

In some embodiments, an oxidation partner may further be provided, leading to the disulfide oxidation. An oxidation partner selected from the group consisting of cystamine glutathione disulfide (GSSG) and cystine may be provided. In some embodiments, cystamine glutathione disulfide (GSSG) may be provided as an oxidation partner.

### Overall Reaction

In a first aspect of the invention and according to the definitions as set out above, a method for producing a peptide or protein comprising a C-terminal α-amide of formula IV, following Scheme 1

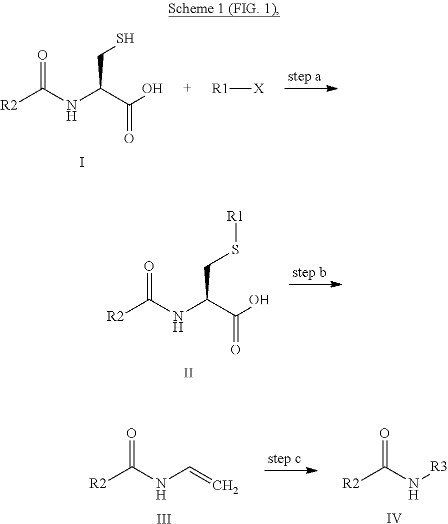

wherein R2 is a polypeptide,
R1-X is a photolabeling agent, wherein R1 is a photolabel and X is a leaving group,
R3 is selected from the group consisting of hydrogen, methyl and ethyl, comprising the steps of
   Step (a). coupling a peptide or protein comprising a C-terminal cysteine of formula I with a photolabel (R1) to obtain a peptide-photolabel conjugate of formula II, and
   Step (b). photochemical conversion of the resulting peptide-photolabel conjugate of formula II to obtain a C-terminal enamide of formula III, and
   Step (c). cleavage of the resulting C-terminal enamide of formula III to obtain the C-terminal α-amide of formula IV,
may be provided.

In some embodiments, R3 may be selected from the group consisting of hydrogen and ethyl. In another embodiment, R3 may be hydrogen.

Also, or alternatively, in a second aspect of the invention, a method for producing a peptide or protein comprising a C-terminal α-amide of formula IV following Scheme 2,

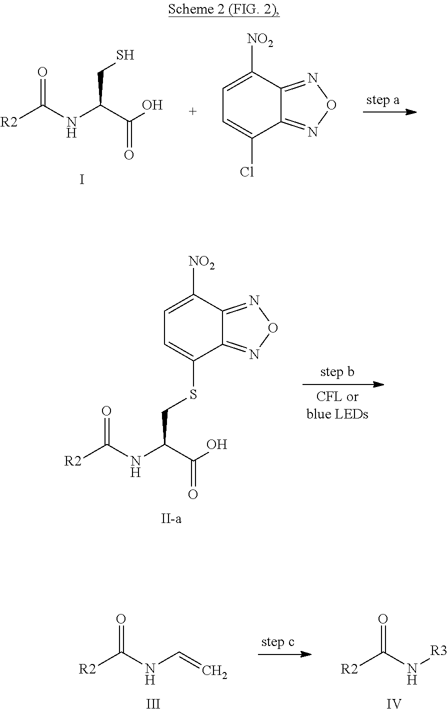

wherein R2 is a polypeptide,
R3 is hydrogen,
comprising the steps of
   Step (a). coupling a peptide or protein comprising a C-terminal cysteine of formula I with 4-chloro-7-nitrobenzofurazan (Chem. 2) to obtain a peptide-photolabel conjugate of formula II-a, and
   Step (b). photochemical conversion of the resulting peptide-photolabel conjugate of formula II-a to obtain a C-terminal enamide of formula III, and
   Step (c). cleavage of the resulting C-terminal enamide of formula III to obtain the C-terminal α-amide of formula IV, may be provided.

In yet another aspect, a method for producing a peptide or protein comprising a C-terminal a-amide of formula IV following Scheme 2,

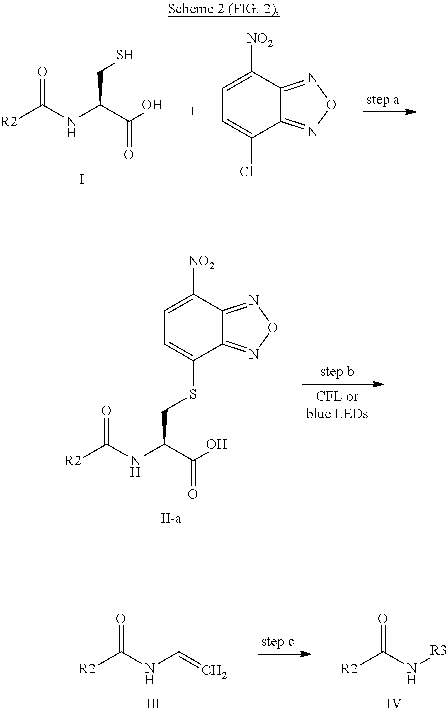

wherein R2 is a polypeptide comprising an amino acid sequence as set out in SEQ ID No. 16, R3 is hydrogen, comprising the steps of
   Step (a). coupling a peptide or protein comprising a C-terminal cysteine amidation tag (formula I) with 4-chloro-7-nitrobenzofurazan to obtain a peptide-photolabel conjugate (formula II-a);
   Step (b). irradiating the peptide-photolabel conjugate (formula II-a) with light having a wavelength between 400-450 nm to obtain a C-terminal enamide (formula III) via photochemical conversion; and
   Step (c). cleaving the C-terminal enamide (formula III) to obtain the C-terminal α-amide (formula IV).

### Purification and Characterisation

The purification of the C-terminal α-amide may be performed in a batch procedure. The purification may be performed by continuous precipitation.

The reaction may be monitored by Liquid Chromatography Mass Spectrometry (LC-MS).

The purification of the C-terminal α-amide may be performed via High Performance Liquid Chromatography (HPLC) or Ultra Filtration Diafiltration (UFDF). The purification of the C-terminal α-amide may be performed via High Performance Liquid Chromatography (HPLC). The purification of the C-terminal α-amide may be performed via Ultra Filtration Diafiltration (UFDF).

Reaction samples may be analysed by UPLC-MS analysis. In some embodiments, the reaction samples may be analysed using extracted ion chromatography. In some embodiments, the reaction may be monitored by LC-MS.

Unless otherwise indicated, terms presented in singular form herein generally also include the plural situation.

The term “compound” is used herein to refer to a molecular entity, and “compounds” may thus have different structural elements besides the minimum element defined for each compound or group of compounds.

Also described herein are compounds and methods in which open ended terms like “comprises” and “comprising” and closed ended terms such as “consists of”, “consisting of”, and the like, may be used.

Where the plural form is used for compounds, starting materials, intermediates, salts and the like, this intends to mean one (preferred) or more single compound(s), salt(s), intermediate(s) or the like, where the singular or the indefinite article (“a”, “an”) is used, this does not intend to exclude the plural, but only preferably means “one”.

### Particular Embodiments

1. A method for producing a peptide or protein comprising a C-terminal α-amide of formula IV, following Scheme 1

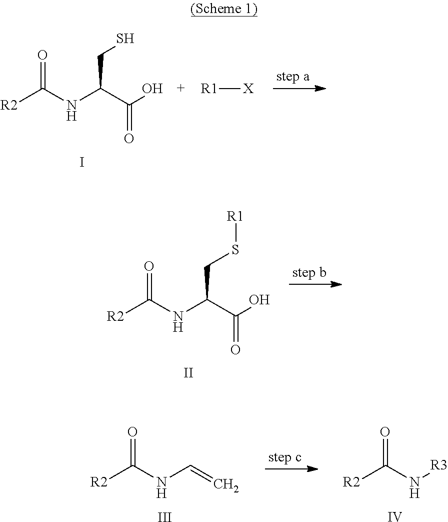

wherein R2 is a polypeptide,
R1-X is a photolabeling agent, wherein R1 is a photolabel and X is a leaving group,
R3 is selected from the group consisting of hydrogen, methyl and ethyl, comprising the steps of
   Step (a). coupling a peptide or protein comprising a C-terminal cysteine amidation tag of formula I with the photolabel (R1) to obtain a peptide-photolabel conjugate of formula II;
   Step (b). irradiating the peptide-photolabel conjugate of formula II to obtain a C-terminal enamide of formula III via a photochemical conversion; and
   Step (c). cleaving the C-terminal enamide of formula III to obtain the C-terminal a-amide of formula IV.
2. The method according to embodiment 1, wherein R3 is selected from the group consisting of hydrogen and ethyl.
3. The method according to any of the preceding embodiments, wherein R3 is hydrogen.
4. The method according to any of the preceding embodiments, where R2 comprises an amino acid sequence as set out in any one of SEQ. ID NO.: 3, 4, 5, 6, 7, 8, 9, 10, 11, 12, 13, 14, 16, 17.
5. The method according to any of the preceding embodiments, wherein R2 consists of an amino acid sequence as set out in any one of SEQ. ID NO.: 3, 4, 5, 6, 7, 8, 9, 10, 11, 12, 13, 14, 16, 17.
6. The method according to any of the preceding embodiments, wherein R2 is an amino acid sequence as set out in any one of SEQ. ID NO. 3-14 and 16-17.
7. The method according to any of the preceding embodiments, wherein R2 is an amino acid sequence as set out in any one of SEQ. ID NO.: 3, 4, 5, 16, 17.
8. The method according to any of the preceding embodiments, wherein R2 comprises an amino acid sequence as set out in SEQ ID No. 16.
9. The method according to any of the preceding embodiments, wherein R2 further comprises a N-terminal extension.
10. The method according to any of the preceding embodiments, wherein R2 further comprises a N-terminal extension having between 1-60 amino acids.
11. The method according to any of the preceding embodiments, wherein R2 further comprises a N-terminal extension having between 1-50 amino acids.
12. The method according to any of the preceding embodiments, wherein R2 further comprises a N-terminal extension having between 1-40 amino acids.
13. The method according to any of the preceding embodiments, wherein R2 further comprises a N-terminal extension having between 1-20 amino acids.
14. The method according to any of the preceding embodiments, wherein R2 further comprises a N-terminal extension having between 1-15 amino acids.
15. The method according to any of the preceding embodiments, wherein R2 further comprises a N-terminal extension having between 5-15 amino acids.
16. The method according to any of the preceding embodiments, wherein R2 further comprises a N-terminal extension having between 14 amino acids.
17. The method according to any of the preceding embodiments, wherein R2 further comprises a N-terminal extension according to SEQ ID No. 18.
18. The method according to any of the preceding embodiments, where R2 is an amino acid sequence as set out in SEQ ID No. 17.
19. The method according to any of the preceding embodiments, wherein the method is used in the preparation of a glucagon-like peptide-1 (GLP-1) peptide.
20. The method according to any one of embodiments 1-18, wherein the method is used in the preparation of an amylin receptor agonist.
21. The method according to embodiment 20 wherein the amylin receptor agonist is Pramlintide.
22. The method according to any one of embodiments 1-18, wherein the method is used in the preparation of a glucose-dependent insulinotropic polypeptide (GIP) peptide.
23. The method according to any one of embodiments 1-18, wherein the method is used in the preparation of a GIP and GLP-1 receptor co-agonist.
24. The method of embodiment 23, wherein the GIP and GLP-1 receptor co-agonist is Tirzepatide.
25. The method according to any one of embodiments 1-18, wherein the method is used in the preparation of urocortin-2 (UCN2) peptide.
26. The method according to any one of embodiments 1-18, wherein the method is used in the preparation of pancreatic pYY (3-36) peptide.
27. The method according to any one of embodiments 1-18, wherein the method is used in the preparation of pancreastatin inhibitor peptide-8 (PSTi8) peptide.
28. The method according to any one of embodiments 1-18, wherein the method is used in the preparation of luteinizing hormone-releasing hormone (LHRH) agonist.
29. The method according to any one of embodiments 1-18, wherein the method is used in the preparation of gastrin releasing peptide.
30. The method according to any one of embodiments 1-18, wherein the method is used in the preparation of Cosyntropin.
31. The method according to any one of embodiments 1-18, wherein the method is used in the preparation of a QRF-amide peptide.
32. The method according to any one of embodiments 1-18, wherein the method is used in the preparation of a GLP-1 receptor-neuropeptide Y receptor 2 co-agonist.
33. The method according to embodiment 32, wherein the GLP-1 receptor-neuropeptide Y receptor 2 co-agonist is EP45.
34. The method according to any one of embodiments 1-18, wherein the method is used in the preparation of Bulevirtide.
35. The method according to any one of embodiments 1-18, wherein the method is used in the preparation of osteocrin (OSTN) peptide.
36. The method according to any one of embodiments 1-18, wherein the method is used in the preparation of Enfuvirtide.
37. The method according to any one of embodiments 1-18, wherein the method is used in the preparation of a GLP-1 receptor-amylin receptor co-agonist.
38. The method according to any one of embodiments 1-18 and 37, wherein the method is used in the preparation of a GLP-1 receptor-amylin receptor co-agonist, the GLP-1 receptor-amylin receptor co-agonist comprising an amino acid sequence as set out in SEQ ID No. 16.
39. The method according to any one of embodiments 1-18 and 37-38, wherein the GLP-1 receptor-amylin receptor co-agonist is represented by SEQ ID No. 16.
40. The method according to any one of embodiments 1-18, wherein the method is used in the preparation of a glucagon-like peptide-1 (GLP-1) peptide, amylin receptor agonist such as Pramlintide, glucose-dependent insulinotropic polypeptide (GIP) peptide, GLP-1 receptor co-agonist such as Tirzepatide, urocortin-2 (UCN2) peptide, pancreatic pYY (3-36) peptide, pancreastatin inhibitor peptide-8 (PSTi8) peptide, luteinizing hormone-releasing hormone (LHRH) agonist, gastrin releasing peptide (GRP) peptide, adrenocorticotropic hormones (ACTH) such as Cosyntropin, QRF-amide peptide, GLP-1 receptor-neuropeptide Y receptor 2 co-agonist such as EP45, entry inhibitors such as Bulevirtide, a natriuretic peptide such as osteocrin (OSTN) peptide, antiretroviral drugs such as Enfuvirtide or GLP-1 receptor-amylin receptor co-agonist such as a GLP-1 receptor-amylin receptor co-agonist comprising the amino acid sequence as set out in SEQ ID No. 16.
41. The method according to any one of embodiments 1-18 and 40, wherein the method is used in the preparation of a glucagon-like peptide-1 (GLP-1) peptide, pancreatic pYY (3-36) peptide, pancreastatin inhibitor peptide-8 (PSTi8) peptide, luteinizing hormone-releasing hormone (LHRH), gastrin releasing peptide (GRP) peptide, Cosyntropin, QRF-amide peptide, GLP-1 receptor-neuropeptide Y receptor 2 co-agonist EP45, Bulevirtide, osteocrin (OSTN) peptide, Enfuvirtide, GLP-1 receptor-amylin receptor co-agonist comprising the amino acid sequence as set out in SEQ ID No. 16 or amylin receptor agonist such as Pramlintide.
42. The method according to any one of embodiments 1-18 and 40-41, wherein the method is used in the preparation of a glucagon-like peptide-1 (GLP-1) peptide, pancreatic pYY (3-36) peptide, pancreastatin inhibitor peptide-8 (PSTi8) peptide, GLP-1 receptor-amylin receptor co-agonist comprising the amino acid sequence as set out in SEQ ID No. 16 or amylin receptor agonist such as Pramlintide.
43. The method according to any one of the preceding embodiments, wherein the method is used in the preparation of Pramlintide.
44. The method according to any one of embodiments 1-18 and 40-42, wherein the method is used in the preparation of a GLP-1 receptor-amylin receptor co-agonist comprising the amino acid sequence as set out in SEQ ID No. 16.
45. The method according to any one of embodiments 1-18 and 40-42, wherein the method is used in the preparation of a precursor GLP-1 receptor-amylin receptor co-agonist comprising the amino acid sequence as set out in SEQ ID No. 16.
46. The method according to any of the preceding embodiments, wherein the photolabeling agent (R1-X) is selected from the group consisting of 3-Bromo-1H-pyrrole-2,5-dione, 4-chloro-7-nitrobenzofurazan, 2-bromo-1,4-naphthoquinone, 1-fluoro-2,4-dinitrobenzene and 4-fluoro-7-sulfamoylbenzofurazan.
47. The method according to any of the preceding embodiments, wherein the photolabeling agent (R1-X) is selected from the group consisting of 3-Bromo-1H-pyrrole-2,5-dione (Chem. 1) or 4-chloro-7-nitrobenzofurazan (Chem. 2):

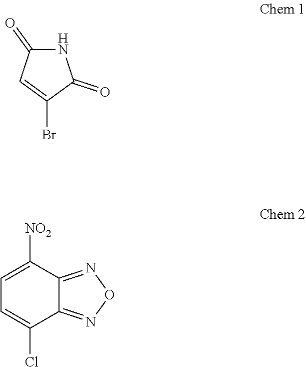 [SMILES: O=C1C=C(Br)C(=O)N1 ; O=[N+]([O-])c1ccc(Cl)c2nonc12]

48. The method according to any of the preceding embodiments, wherein the photolabeling agent is 3-Bromo-1H-pyrrole-2,5-dione (Chem. 1):

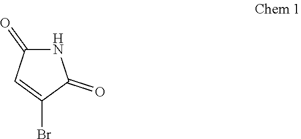

49. The method according to any of the preceding embodiments, wherein the photolabeling agent is 4-chloro-7-nitrobenzofurazan (Chem. 2):

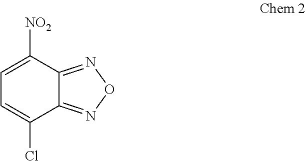 [SMILES: O=[N+]([O-])c1ccc(Cl)c2nonc12]

50. The method according to any of the preceding embodiments, wherein the method takes place in an aqueous reaction buffer.
51. The method according to any of the preceding embodiments, wherein step (a) of the method takes place in an aqueous reaction buffer.
52. The method according to any of the preceding embodiments, wherein step (a) of the method takes place in an aqueous reaction buffer, the aqueous buffer selected from the group consisting of bis-tris methane, tris, triethanolamine and phosphate.
53. The method according to any of the preceding embodiments, wherein step (a) of the method takes place in an aqueous reaction buffer of bis-tris methane.
54. The method according to any of the preceding embodiments, wherein step (a) of the method takes place in an aqueous reaction buffer of about 25 mM bis-tris methane.
55. The method according to any of the preceding embodiments, wherein step (a) of the method takes place in an aqueous reaction buffer of tris.
56. The method according to any of the preceding embodiments, wherein step (a) of the method takes place in an aqueous reaction buffer of triethanolamine.
57. The method according to any of the preceding embodiments, wherein step (a) of the method takes place in an aqueous reaction buffer of phosphate.
58. The method according to any of the preceding embodiments, wherein step (a) of the method takes place in an aqueous reaction buffer at pH 4-9.
59. The method according to any of the preceding embodiments, wherein step (a) of the method takes place in an aqueous reaction buffer at pH 6-8.
60. The method according to any of the preceding embodiments, wherein step (a) of the method takes place in an aqueous reaction buffer at pH 6-7.
61. The method according to any of the preceding embodiments, wherein step (a) of the method takes place in an aqueous reaction buffer at pH 6.3-7.
62. The method according to any of the preceding embodiments, wherein step (a) of the method takes place in an aqueous reaction buffer at pH 6.3-6.4.
63. The method according to any of the preceding embodiments, wherein step (a) of the method takes place in an aqueous reaction buffer at about pH 6.4.
64. The method according to any one of embodiments 1-42, wherein step (a) of the method takes place in an aqueous reaction buffer at about pH 6.3.
65. The method according to any one of embodiments 1-41, wherein step (a) of the method takes place in an aqueous reaction buffer at about pH 7.
66. The method according to any of the preceding embodiments, wherein step (a) of the method takes place in an aqueous reaction buffer with glycine.
67. The method according to any of the preceding embodiments, wherein step (a) of the steps of the method take place in an aqueous reaction buffer with about 50 mM of glycine.
68. The method according to any of the preceding embodiments, wherein between 1 and 5 equivalents of the photolabel relative to the peptide in step (a) are provided.
69. The method according to any of the preceding embodiments, wherein between 1 and 3 equivalents of the photolabel relative to the peptide in step (a) are provided.
70. The method according to any of the preceding embodiments, wherein between 1.5 and 2.5 equivalents of the photolabel relative to the peptide in step (a) are provided.
71. The method according to any of the preceding embodiments, wherein between 1.8 and 2.2 equivalents of the photolabel relative to the peptide in step (a) are provided.
72. The method according to any of the preceding embodiments, wherein 2 equivalents of the photolabel relative to the peptide in step (a) are provided.
73. The method according to any of the preceding embodiments, wherein a source of light for irradiating the peptide-photolabel conjugate is a compact fluorescent lamp (CFL).
74. The method according to any of the preceding embodiments, wherein the source of light for irradiating the peptide-photolabel conjugate is a at least one light emitting diode (LED).
75. The method according to any of the preceding embodiments, wherein the source of light for irradiating the peptide-photolabel conjugate is a plurality of light emitting diodes (LEDs).
76. The method according to any of the preceding embodiments, wherein the source of light for irradiating the peptide-photolabel conjugate is a monochromatic LED.
77. The method according to any of the preceding embodiments, wherein the source of light for irradiating the peptide-photolabel conjugate has a wavelength providing white light.
78. The method according to any of the preceding embodiments, wherein the source of light for irradiating the peptide-photolabel conjugate has a wavelength between 300-700 nm.
79. The method according to any of the preceding embodiments, wherein the source of light for irradiating the peptide-photolabel conjugate has a wavelength between 365-550 nm.
80. The method according to any of the preceding embodiments, wherein the source of light for irradiating the peptide-photolabel conjugate has a wavelength between 365-500 nm.
81. The method according to any of the preceding embodiments, wherein the source of light for irradiating the peptide-photolabel conjugate has a wavelength between 365-450 nm.
82. The method according to any of the preceding embodiments, wherein the source of light for irradiating the peptide-photolabel conjugate has a wavelength of 365 nm.
83. The method according to any of the preceding embodiments, wherein the source of light for irradiating the peptide-photolabel conjugate has a wavelength between 400-500 nm.
84. The method according to any of the preceding embodiments, wherein the source of light for irradiating the peptide-photolabel conjugate has a wavelength between 400-450 nm.
85. The method according to any of the preceding embodiments, wherein the source of light for irradiating the peptide-photolabel conjugate has a wavelength of about 405 nm.
86. The method according to any of the preceding embodiments, wherein the source of light for irradiating the peptide-photolabel conjugate has a wavelength of about 430 nm.
87. The method according to any of the preceding embodiments, wherein the source of light for irradiating the peptide-photolabel conjugate has a wavelength of about 450 nm.
88. The method according to any of the preceding embodiments, wherein the enamide is cleaved by acid mediated hydrolysis.
89. The method according to any of the preceding embodiments, wherein the enamide is hydrogenated.
90. The method according to any of the preceding embodiments, wherein the enamide is cleaved by acid catalysed, oxidizing agent mediated hydrolysis or by an Inverse Electron Demand Diels-Alder reaction.
91. The method according to any of the preceding embodiments, wherein the enamide is cleaved by acid catalysed or by oxidizing agent mediated hydrolysis.
92. The method according to any of the preceding embodiments, wherein the enamide is cleaved by acid catalysed hydrolysis.
93. The method according to any of the preceding embodiments, wherein the enamide is cleaved by oxidizing agent mediated hydrolysis.
94. The method according to any of the preceding embodiments, wherein the enamide is cleaved by an Inverse Electron Demand Diels-Alder reaction.
95. The method according to any of the preceding embodiments, further comprising a step of adding a cleavage reagent for cleaving the enamide.
96. The method according to any of the preceding embodiments, further comprising a step of adding a cleavage reagent for cleaving the enamide wherein the cleavage reagent is selected from the group consisting of trifluoroacetic acid (TFA), hydrochloric acid (HCl), Sulfuric acid (H₂SO₄), tosylic acid (TsOH), phosphoric acid (H₃PO₄), oxalic acid, 6,6′-(1,2,4,5-tetrazine-3,6-diyl) dinicotinic acid.
97. The method according to any of the preceding embodiments, wherein the cleavage reagent is selected from the group consisting of trifluoroacetic acid (TFA), hydrochloric acid (HCl), Sulfuric Acid (H₂SO₄), tosylic acid (TsOH), phosphoric acid (H₃PO₄), oxalic acid and 6,6′-(1,2,4,5-tetrazine-3,6-diyl) dinicotinic acid.
98. The method according to any of the preceding embodiments, wherein the cleavage reagent is selected from the group consisting of trifluoroacetic acid (TFA), hydrochloric acid (HCl), Sulfuric Acid (H₂SO₄), tosylic acid (TsOH), phosphoric acid (H₃PO₄) and 6,6′-(1,2,4,5-tetrazine-3,6-diyl) dinicotinic acid.
99. The method according to any of the preceding embodiments, wherein the cleavage reagent is trifluoroacetic acid (TFA).
100. The method according to any of the preceding embodiments, wherein the cleavage reagent is 2-8% v/v trifluoroacetic acid (TFA).
101. The method according to any of the preceding embodiments, wherein the cleavage reagent is 4-6% v/v trifluoroacetic acid (TFA).
102. The method according to any of the preceding embodiments, wherein the cleavage reagent is 5% v/v trifluoroacetic acid (TFA).
103. The method according to any of the preceding embodiments, wherein the cleavage reagent is hydrochloric acid (HCl).
104. The method according to any of the preceding embodiments, wherein the cleavage reagent is sulfuric acid (H₂SO₄).
105. The method according to any of the preceding embodiments, wherein the cleavage reagent is phosphoric acid (H₃PO₄).
106. The method according to any of the preceding embodiments, wherein the cleavage reagent is 0.5-2 M phosphoric acid (H₃PO₄).
107. The method according to any of the preceding embodiments, wherein the cleavage reagent is 0.5-1.5 M phosphoric acid (H₃PO₄).
108. The method according to any of the preceding embodiments, wherein the cleavage reagent is 0.7-1.3 M phosphoric acid (H₃PO₄).
109. The method according to any of the preceding embodiments, wherein the cleavage reagent is 0.8-1.2 M phosphoric acid (H₃PO₄).
110. The method according to any of the preceding embodiments, wherein the cleavage reagent is about 1 M phosphoric acid (H₃PO₄).
111. The method according to any of the preceding embodiments, wherein the cleavage reagent is tosylic acid (TsOH).
112. The method according to any of the preceding embodiments, wherein the cleavage reagent is 6,6′-(1,2,4,5-tetrazine-3,6-diyl) dinicotinic acid.
113. The method according to any of the preceding embodiments, wherein at least part of the method is performed in a batch procedure.
114. The method according to any of the preceding embodiments, wherein the enamide cleavage is performed at a temperature between 0 and 80 degrees Celsius.
115. The method according to any of the preceding embodiments, wherein the enamide cleavage is performed at a temperature between 20 to 60 degrees Celsius.
116. The method according to any of the preceding embodiments, wherein the enamide cleavage is performed at a temperature between 20 and 40 degrees Celsius.
117. The method according to any of the preceding embodiments, wherein the enamide cleavage is performed at a temperature between 20 and 26 degree Celsius.
118. The method according to any of the preceding embodiments, wherein the enamide cleavage is performed at a temperature between 20 and 23 degrees Celsius.
119. The method according to any of the preceding embodiments, wherein the enamide cleavage is performed at a temperature of 20 degrees Celsius.
120. The method according to any of the preceding embodiments, wherein the enamide cleavage is performed at a temperature of 23 degrees Celsius.
121. The method according to any of the preceding embodiments, wherein the enamide cleavage is performed at room temperature.
122. The method according to any one of embodiments 1-116, wherein the enamide cleavage is performed at a temperature between 30 and 40 degrees Celsius.
123. The method according to any one of embodiments 1-116 and 122, wherein the enamide cleavage is performed at a temperature of about 37 degrees Celsius.
124. The method according to any one of the preceding embodiments, wherein at least part of the method is performed in a flow reactor.
125. The method according to any of the preceding embodiments, wherein the irradiating step is performed in a flow reactor.
126. The method according to any of the preceding embodiments, wherein only the irradiating step is performed in a flow reactor.
127. The method according to any one of embodiments 124-126, wherein the temperature of the flow reactor is 20 to 60 degrees Celsius.
128. The method according to any one of embodiments 124-127, wherein the temperature of the flow reactor is 20 to 40 degrees Celsius.
129. The method according to any one of embodiments 124-128, wherein the temperature of the flow reactor is 20 to 30 degrees Celsius.
130. The method according to any one of embodiments 124-129, wherein the temperature of the flow reactor is 25 to 30 degrees Celsius.
131. The method according to any one of embodiments 124-130, wherein the temperature of the flow reactor is about 26 degrees Celsius.
132. The method according to any one of embodiments 124-131, wherein a flow rate of the flow reactor is 0.200 to 50.00 mL/min.
133. The method according to any one of embodiments 124-132, wherein the flow rate of the flow reactor is 1.00 to 25.00 mL/min.
134. The method according to any one of embodiments 124-133, wherein the flow rate of the flow reactor is 5.00 to 10.00 mL/min.
135. The method according to any one of embodiments 124-134, wherein the flow rate of the flow reactor is 6.00 to 9.00 mL/min.
136. The method according to any one of embodiments 124-135, wherein the flow rate of the flow reactor is about 8.00 mL/min.
137. The method according to any one of embodiments 124-136, wherein a reactor volume of the flow reactor is 1 to 10 mL.
138. The method according to any one of embodiments 124-137, wherein the reactor volume of the flow reactor is 2 to 5 mL.
139. The method according to any one of embodiments 124-138, wherein the reactor volume of the flow reactor is 1 to 3 mL.
140. The method according to any one of embodiments 124-139, wherein the reactor volume of the flow reactor is about 2 mL.
141. The method according to any one of embodiments 124-140, wherein the reactor volume of the flow reactor is about 2.7 mL.
142. The method according to any one of embodiments 124-141, wherein a residence time in the flow reactor is 0.04 to 50 min.
143. The method according to any one of embodiments 124-142, wherein the residence time in the flow reactor is 0.1 to 10 min.
144. The method according to any one of embodiments 124-143, wherein the residence time in the flow reactor is 0.1 to 1 min.
145. The method according to any one of embodiments 124-144, wherein the residence time in the flow reactor is 0.1 to 0.5 min.
146. The method according to any one of embodiments 124-145, wherein the residence time in the flow reactor is about 0.25 min.
147. The method according to any one of embodiments 124-146, wherein the flow reactor is a Vapourtec UV-150 photochemical reactor.
148. The method according to any one of embodiments 124-146, wherein the flow reactor is a Corning® Lab Photo Reactor.
149. The method according to any one of embodiments 124-148, wherein the light source irradiates at a radiant power between 3-150 watts.
150. The method according to any one of embodiments 124-149, wherein the light source irradiates at a radiant power between 5-100 watts.
151. The method according to any one of embodiments 124-150, wherein the light source irradiates at a radiant power between 10-80 watts.
152. The method according to any one of embodiments 124-151, wherein the light source irradiates at a radiant power between 9-24 watts.
153. The method according to any of the preceding embodiments, wherein the reaction is performed in a single reaction vessel.
154. The method according to any of the preceding embodiments, further comprising adding a surfactant.
155. The method according to any of the preceding embodiments, further comprising adding a surfactant selected from the group consisting of sodium dodecyl sulfate (SDS), sodium desoxycholate (SDC), 3-[(3-cholamidopropyl) dimethylammonio]-1-propanesulfonate (CHAPS), Triton™ X-100, Tween-20, Tetrapropylammonium hydroxide (TAPH-40) and sodium octanoate.
156. The method according to any of the preceding embodiments, further comprising adding a surfactant, wherein the surfactant is sodium dodecyl sulfate (SDS).
157. The method according to any of the preceding embodiments, further comprising a surfactant, wherein the surfactant is 3-[(3-adding cholamidopropyl) dimethylammonio]-1-propanesulfonate (CHAPS).
158. The method according to any of the preceding embodiments, further comprising adding a surfactant, wherein the surfactant is sodium octanoate.
159. The method according to any of the preceding embodiments, further comprising adding 40-80 mM of a surfactant.
160. The method according to any of the preceding embodiments, further comprising adding 50-70 mM of a surfactant.
161. The method according to any of the preceding embodiments, further comprising adding 55-65 mM of a surfactant.
162. The method according to any of the preceding embodiments, further comprising adding about 60 mM of a surfactant.
163. The method according to any of the preceding embodiments, further comprising the steps of
   coupling a cysteine of the peptide R2 with the photolabel R1 forming a photolabel-protected cysteine prior to step (b); and
   releasing the photolabel-protected cysteine from the peptide R2 after step (b).
164. The method according to embodiment 163, wherein a nucleophilic sulfide and an oxidation partner are provided.
165. The method according to any one of embodiments 163-164 further comprising adding a nucleophilic sulfide for releasing the photolabel-protected cysteine from the peptide R2.
166. The method according to any one of embodiments 163-165, further comprising adding a nucleophilic sulfide for releasing the photolabel-protected cysteine from the peptide R2, wherein the nucleophilic sulfide is selected from the group consisting of cysteamine, cysteine, dithiothreitol (DTT), 3,6-dioxa-1,8-octanedithiol (DODT), 2-mercaptoethanol, glutathione (GSH) and acetylcysteine.
167. The method according to any one of embodiments 163-166, further comprising adding a nucleophilic sulfide for releasing the photolabel-protected cysteine from the peptide R2, wherein the nucleophilic sulfide is cysteamine.
168. The method according to any one of embodiments 163-167, further comprising forming a disulfide bond in peptide R2 after releasing the photolabel-protected cysteines.
169. The method according to any one of embodiments 163-168, further comprising adding an oxidation partner.
170. The method according to any one of embodiments 163-169, further comprising adding an oxidation partner selected from the group consisting of cystamine glutathione disulfide (GSSG) and cystine.
171. The method according to any one of embodiments 163-170, further comprising adding an oxidation partner, wherein the oxidation partner is cystamine glutathione disulfide (GSSG).
172. The method according to any one of embodiments 163-171, further comprising adding between 0.5 and 3 additional equivalents of the photolabel relative to the peptide at step (a) for each disulfide bond to be formed.
173. The method according to any one of embodiments 163-172, further comprising adding between 1 and 2.5 additional equivalents of the photolabel relative to the peptide at step (a) for each disulfide bond to be formed.
174. The method according to any one of embodiments 163-173, further comprising adding between 1.5 and 2.5 additional equivalents of the photolabel relative to the peptide at step (a) for each disulfide bond to be formed.
175. The method according to any one of embodiments 163-174, further comprising adding about 2 additional equivalents of the photolabel relative to the peptide at step (a) for each disulfide bond to be formed.
176. The method according to any of the preceding embodiments, further comprising a step of purifying the C-terminal α-amide, wherein the purification of the C-terminal a-amide is performed in a batch procedure or by continuous precipitation.
177. The method according to any of the preceding embodiments, further comprising a step of purifying the C-terminal α-amide, wherein the purification of the C-terminal a-amide is performed via High Performance Liquid Chromatography (HPLC) or Ultra Filtration Diafiltration (UFDF).
178. The method according to any of the preceding embodiments, further comprising a step of purifying the C-terminal α-amide, wherein the purification of the C-terminal a-amide is performed via High Performance Liquid Chromatography (HPLC).
179. The method according to any of the preceding embodiments, further comprising a step of purifying the C-terminal α-amide, wherein the purification of the C-terminal a-amide is performed via Ultra Filtration Diafiltration (UFDF).
180. A method for producing a peptide or protein comprising a C-terminal α-amide of formula IV following Scheme 2,

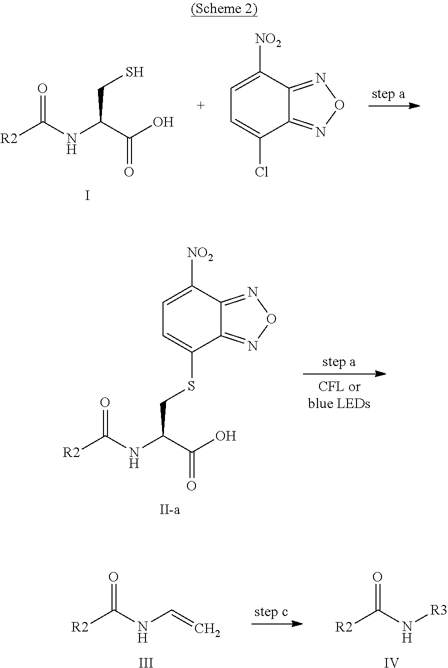

wherein R2 is a polypeptide,
R3 is hydrogen,
comprising the steps of
   Step (a). coupling a peptide or protein comprising a C-terminal cysteine amidation tag of formula I with 4-chloro-7-nitrobenzofurazan to obtain a peptide-photolabel conjugate of formula II-a;
   Step (b). irradiating the peptide-photolabel conjugate of formula II-a to obtain a C-terminal enamide of formula III via photochemical conversion; and
   Step (c). cleaving the C-terminal enamide of formula III to obtain the C-terminal a-amide of formula IV.
181. A method for producing a peptide or protein comprising a C-terminal α-amide of formula IV following Scheme 2,

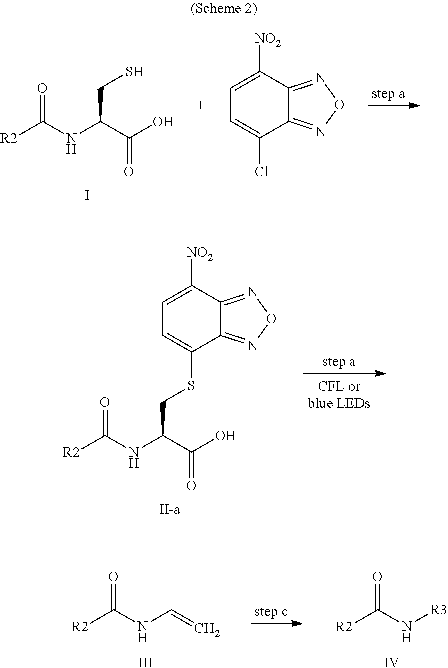

wherein R2 is a polypeptide comprising an amino acid sequence as set out in SEQ ID No. 16,
R3 is hydrogen,
comprising the steps of
   Step (a). coupling a peptide or protein comprising a C-terminal cysteine amidation tag of formula I with 4-chloro-7-nitrobenzofurazan to obtain a peptide-photolabel conjugate of formula II-a;
   Step (b). irradiating the peptide-photolabel conjugate of formula II-a with light having a wavelength between 400-450 nm to obtain a C-terminal enamide of formula III via photochemical conversion; and
   Step (c). cleaving the C-terminal enamide of formula III to obtain the C-terminal a-amide of formula IV.
182. A method of producing a pharmaceutical composition according to any of the embodiments 1-181.
183. A method of producing a pharmaceutical composition, comprising the steps of
   a. producing a compound of formula IV, according to any of the embodiments 1-181,
   b. preparing said pharmaceutical composition using the compound of formula IV obtained in a.

### METHODS AND EXAMPLES

### List of Abbreviations

Aq., aq. Aqueous
Ac Acetyl
CFL Compact Fluorescent Lamp
CLND Chemiluminescent Nitrogen Detection
DIC diisopropyl-carbodiimide
DODT 3,6-dioxa-1,8-octanedithiol
DMF N, N-Dimethylformamide
DMSO Dimethyl sulfoxide
EIC Extracted Ion Chromatogram
Et Ethyl
Eq. Equivalents
Fmoc fluorenylmethoxycarbonyl protecting group
h Hour(s)
HRMS High Resolution Mass Spectrometry
HPLC High Performance Liquid Chromatography
HC Hydrochloric acid
i-Pr iso-propyl
IEDDA Inverse Electron Demand Diels-Alder
LCMS Liquid Chromatography Mass Spectrometry
LEDs Light-emitting Diodes
Me Methyl
Min Minute(s)
MWCO Molecular Weight Cut Off
NBD-CI 4-chloro-7-nitrobenzofurazan
NHS N-hydroxysuccimide
NMP N-Methyl-2-pyrrolidone
RP-HPLC Reverse-Phase High Performance Liquid Chromatography
SPPS Solid Phase Peptide Synthesis
TFA Trifluoroacetic acid
TIPS Triisopropylsilane
UPLC Ultra Performance Liquid Chromatography
UPLC-MS Ultra Performance Liquid Chromatography Mass Spectrometry

Note that in the Examples, the numbers of compounds, such as Chem. 1, are different than the numbers representing the compounds in the description and in the embodiments, such as (I) or (II), just to clarify paradigmatically.

### General Methods of Preparation

The synthesis of α-amidated peptides has been performed in three different ways, either via a screening scale procedure, a batch procedure, or via a continuous photo-flow process.

### Reagents

All reagents and solvents were purchased commercially and used as supplied. Orthogonally protected Fmoc-amino acids were purchased from Iris Biotech as pre-weighed cartridges and SPPS resins were purchased from NovaBioChem.

### General Method for Automated SPPS

Automated SPPS was conducted on a Symphony X peptide synthesizer (Gyros Biotechnologies). Peptides were synthesized using standard Fmoc chemistry on pre-loaded Fmoc-amino acid Wang resins (˜0.34 mmol/g loading). Couplings were carried out using 5 equiv. of Fmoc amino acid in DMF, 10 equiv. of diisopropyl-carbodiimide (DIC), 5 equiv. of 2,4,6-collidine and 5 equiv. of ethyl cyanohydroxy-iminoacetate (oxyma). Coupling reactions proceeded at RT for 1.5-4 h with N₂ bubbling for mixing. Fmoc deprotections were performed using 20% piperidine in DMF.

### General Method of Cleavage

Peptide cleavage was conducted in a 90/2.5/2.5/2.5/2.5 solution of TFA/triisopropylsilane (TIPS)/water/thioanisole/3,6-dioxa-1,8-octanedithiol (DODT). The eluted cleavage solution was precipitated in ice cold diethyl ether. The peptide precipitate was pelleted using centrifugation and the pellet washed with additional ice-cold ether.

### General Method of Purification and Quantification

The washed peptide pellet was re-dissolved in either 1:1 acetonitrile/water or neat DMSO, then filtered via a 0.2 μm filter. The resulting solution was loaded onto a preparative RP-HPLC system, either a Waters Prep 150 LC system or an Agilent 1290 Infinity II Autoscale. Preparative LC/MSD system. Both systems were equipped with a Waters XSelect CSH C18 column (19×250 mm, 5 μm) at a flow rate of 20 mL/min [Buffer A: 89.9% water/10% acetonitrile with 0.1% TFA; Buffer B: 89.9% acetonitrile/10% water with 0.1% TFA]. Peptides were resolved using an appropriate solvent gradient. Pure fractions were pooled, evaluated for purity/identity by UPLC/LCMS, peptide content by chemiluminescent nitrogen detection (CLND), and lyophilized to dry powder.

### General Methods of Detection and Characterisation

Analytical UPLC was conducted on a Waters Acquity UPLC system equipped with a Waters BEH C18 column (2.1×150 mm, 1.7 μm) at a flow rate of 0.4 mL/min [Buffer A: 99.95% water with 0.05% TFA; Buffer B: 99.95% acetonitrile with 0.05% TFA; Gradient: hold at 95/5 A:B for 0.5 minutes, gradient from 95/5 A:B to 5/95 A:B over 16 minutes, followed by wash and re-equilibration]. UV absorbance was measured at 214 nm. For LCMS and HRMS analysis, MS detection was obtained using Waters Xevo G2-XS QTOF mass spectrometer in ESI+mode.

### General Reaction Protocols for C-Terminal α-Amidation

The reactions performed in this invention generally follow SCHEME 1 depicted below:

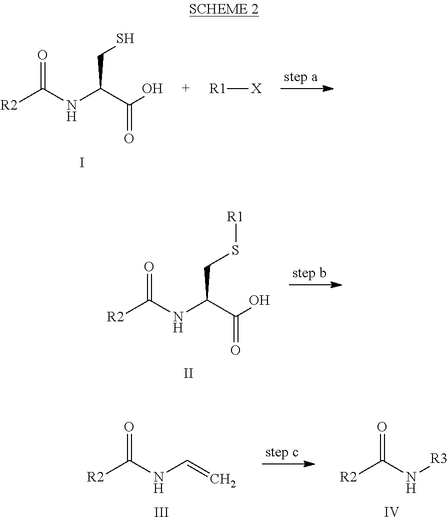

The meaning of the variable substituents in the above formula is the same as in SCHEME (1).

The α-amidation reactions of the present invention have been carried out following three different protocols: Screening Scale, Batch Scale and Flow procedure. They will be explained in further detail below.

### General Protocol for Screening Scale Reaction:

The reaction was initially carried out on a screening scale following Scheme 1, and the procedures of the individual steps ((a), (b) and (c)) are described below in further detail.

Step (a)—Coupling of Peptide or Protein Comprising C-Terminal Cysteine Residue with Photolabel

To minimize non-specific oxidation, aqueous reaction buffer containing 25 mM bis-tris methane pH 6.4 with 50 mM glycine was degassed by bubbling nitrogen gas for 15 minutes. The C-terminal Cys modified peptide was dissolved in reaction buffer to generate a 1 mM stock solution. 25 μL of 1 mM stock was added to a well of a 96-well V-bottom assay plate, along with 65 μL of additional assay buffer. Either 2-bromomaleimide (3-Bromo-1H-pyrrole-2,5-dione, CAS: 98026-79-0) or NBD-chloride (4-chloro-7-nitrobenzofurazan, CAS: 10199-89-0) was dissolved in acetonitrile to produce a 5 mM stock solution. 10 μL of the 5 mM photo conjugate reagent was added to the well (500 μM final concentration, 2 equiv.), and allowed to incubate for 1 hour at room temperature with shaking. If desired, conversion can be monitored by LC-MS.

Step (b)—Photochemical Conversion of the Resulting Peptide-Photolabel Conjugate to Obtain a C-Terminal Enamide:

After 1 hour, the plate was irradiated according to the photo aryl group utilized to generate the C-terminal enamide. For 2-bromomaleimide, a 1.5 meter strip of 365 nm LEDs served as the irradiation source, and the conversion proceeded for 4 hours. A cooling fan was used to maintain the reaction mixture at room temperature. For NBD-chloride, irradiation can be conducted with a handheld white CFL lamp or a strip of 450 nm LEDs. The conversion proceeded for 1 hour at room temperature. If desired, conversion can be monitored by LC-MS.

Step (c)—Cleavage of the Resulting C-Terminal Enamide to Obtain the C-Terminal α-Amide:

### Acidolysis of Enamide to C-Terminal α-Amide Product:

Acidolysis of the enamide proceeded using a range strong acids (Table 2). In general, the acid was added to the reaction mixture from a 10× aqueous stock solution. Trifluoroacetic acid was used for the majority of testing conditions. For TFA, 10 UL of a 50/50 solution of TFA/water was added to the reaction well (5% final TFA concentration) and the plate was shaken at room temperature for up to 24 hours. When additives such as methionine, indole or caffeic acid were utilized, the additive reagent was spiked from a 50× or 100× stock solution into the reaction prior to addition of the acid. Conversion was monitored by LC-MS.

### Enamide Cleavage Using Inverse-Electron Demand Diels-Alder (IEDDA)

The enamide can also be cleaved using IEDDA chemistry with dipyridyl-tetrazine (3,6-Di-2-pyridyl-1,2,4,5-tetrazine, CAS: 1671-87-0). Dipyridyl-tetrazine was dissolved in a mixture of 80% acetonitrile and 20% 125 mM aq. HCl to produce a 12.5 mM stock solution (final concentration of HCl=25 mM, 2 equiv. based on tetrazine conc.). Solubility of the dipyridyl-tetrazine was poor in acetonitrile alone and required addition of acid to protonate pyridyl groups and improve solubility. 10 μL of this stock solution was added to the reaction well (1.25 mM final tetrazine conc., 5 equiv. based on peptide conc.), and the reaction was incubated at 37° C. for 24 hours. Conversion was monitored by LC-MS.

### Analysis of Conversion by UPLC-MS

Reaction samples were directly analyzed by UPLC-MS analysis (Gradient: Hold at 95/5 A/B for 0.5 minutes, gradient from 95/5 A/B to 55/45 A/B over 7 minutes, followed by wash and re-equilibration). In several cases, it was not possible to achieve adequate chromatographic resolution to quantify conversion using integrated UV data at 214 nm. To quantify conversion, the calculated masses of all peptide starting materials, intermediates, side products, and desired products were input to generate an extracted ion chromatogram (EIC). The integrated peaks of these EICs were summed and normalized to 100%, and conversion to desired product calculated based on the normalized integration.

### Protocol for Batch Scale Reaction:

In a vial equipped with a stir bar or a 12.5 mL capacity colorimeter cuvette (catalogue #76016-356), 1.0 equiv of a protein, peptide or polypeptide comprising a C-terminal cysteine residue was dissolved in Bis-tris methane buffer (25 mM, pH 6.3) to a final peptide concentration of 250 uM. A stock solution of (3-Bromo-1H-pyrrole-2,5-dione, CAS: 98026-79-0) or NBD-chloride (4-chloro-7-nitrobenzofurazan, CAS: 10199-89-0) was prepared in 1:1 H₂O:MeCN, and 2 equivalents relative to peptide are added to the reaction vessel for cysteine coupling. The reaction was allowed to stir for 1 hour or until the reaction was complete by LC/MS. The reaction vessel was then irradiated with 365 nm LEDs, a handheld white CFL work light, or a strip of 450 nm blue LEDs over 0.25-4 h. A cooling fan was used to maintain the reaction mixture at room temperature. Once the reaction was complete by LC/MS, trifluoroacetic acid (TFA) was added (5% final TFA concentration) and the reaction was allowed to stir at ambient temperature for 12-24 h. Alternatively, 5 equivalents of dipyridyl-tetrazine (3,6-Di-2-pyridyl-1,2,4,5-tetrazine, CAS: 1671-87-0) was added from a stock solution to give a final concentration of 1.25 mM tetrazine (2.5 mM HCl, dissolved in 80:20 MeCN: H₂O), and the reaction was incubated at 37° C. for 24 hours. Conversion to C-terminal α-amide was monitored by LC-MS.

### Protocol for Batch Scale Reaction of Peptides Containing a Disulfide Bond:

In a vial equipped with a stir bar or a 12.5 mL capacity colorimeter cuvette (catalogue #76016-356), 1.0 equiv of a protein, peptide or polypeptide comprising a C-terminal cysteine residue was dissolved in Bis-tris methane buffer (25 mM, pH 6.3) to a final peptide concentration of 250 uM. A surfactant may be added to the solution to improve the solubility of the intermediates. A concentration of 60 mM surfactant may be preferred. A stock solution of NBD-chloride (4-chloro-7-nitrobenzofurazan, CAS: 10199-89-0) was prepared in 1:1 H₂O:MeCN, and 4 equivalents relative to peptide were added to the reaction vessel for global cysteine coupling. The reaction was allowed to stir for 2 hour or until the reaction was complete by LC/MS. The reaction vessel was then irradiated with a handheld white CFL work light, or a strip of 450 nm blue LEDs over 0.25-4 h. A cooling fan was used to maintain the reaction mixture at room temperature. Once the reaction was complete by LC/MS, a solution of nucleophilic sulfide, e.g. cysteamine (8 equiv) and cystamine (1.2 equiv) in H₂O is added and allowed to stir at ambient temperature for 2 h to liberate the backbone cysteine thiols and oxidize to the disulfide. Trifluoroacetic acid (TFA) was added (5% final TFA concentration) and the reaction was allowed to stir at ambient temperature for 12-24 h. Alternatively, 5 equivalents of dipyridyl-tetrazine (3,6-Di-2-pyridyl-1,2,4,5-tetrazine, CAS: 1671-87-0) may be added from a stock solution to give a final concentration of 1.25 mM tetrazine (2.5 mM HCl, dissolved in 80:20 MeCN: H₂O), and the reaction can be incubated at 37° C. for 24 hours. Conversion to C-terminal α-amide was monitored by LC-MS.

### Example 1: Photolabeling Experiments

Following the general procedure outlined for batch scale α-amidation, a range of photolabels were evaluated, covering a variety of chemical scaffolds and wavelengths of absorption. Reactions were directly analysed by UPLC-MS as outlined above for the conversion to the C-terminal enamide, following photolabel conjugation and irradiation with an appropriate light source. The results are presented in Table 1.

TABLE 1
Coupling of polypeptide with photolabeling agents and photochemical
conversion to enamide (step (a) and (b))

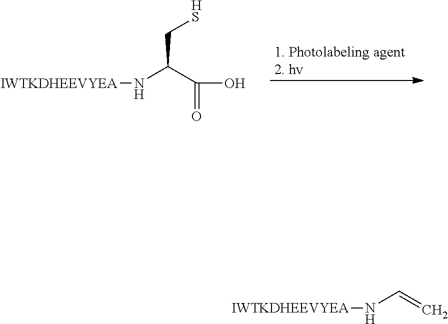

| | | Enamide |
|---|---|---|
| Photolabeling agent | Light Source | Yield [%] |
| 3-Bromo-1H-pyrrole-2,5-dione | 365 nm LEDs | 89 |
| 4-chloro-7-nitrobenzofuran | CFL | 94 |
| 2-bromo-1,4-naphthoquinone | CFL | 58 |
| 1-fluoro-2,4-dinitrobenzene | 365 nm LEDs | 41 |
| 4-fluoro-7-sulfamoylbenzofurazan | CFL | 81 |

Conversion was observed for the photolabeling and conversion of a peptide with an R2 of amino acid sequence as set out in SEQ ID No. 2 containing a C-terminal cysteine to C-terminal enamide. The reaction was performed with structurally diverse photolabeling agents and conversion of the photolabel conjugates was achieved at 365 nm and using visible light.

### Example 2: Enamide Cleavage Experiments

Following the general protocol for screening scale α-amidation, a broad range of acids and tetrazines were evaluated for the cleavage of the enamide to the corresponding C-terminal α-amide (step (c)). The results are presented in Table 2.

TABLE 2
Cleavage of enamide to C-terminal α-amide with acids
or tetrazines (step C)

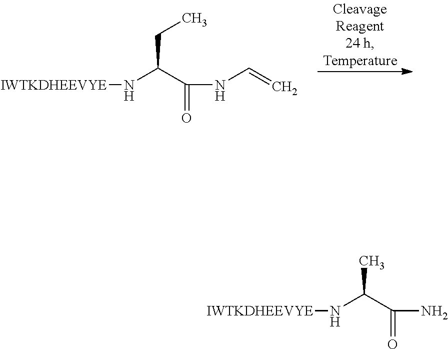

| | | Temperature | Yield |
|---|---|---|---|
| Entry | Enamide Cleavage Reagent | [° C.] | [%] |
| 1 | TFA (5% v/v) | 23 | 64 |
| 2 | HCl (500 mM) | 23 | 65 |
| 3 | H₂SO₄ (500 mM) | 23 | 65 |
| 4 | TsOH (5% w/v) | 23 | 67 |
| 5 | H₃PO₄ (500 mM) | 23 | 37 |
| 6 | Oxalic acid (5% w/v) | 23 | 51 |
| 7 | 3,6-Di-2-pyridyl-1,2,4,5-tetrazine | 37 | 79 |
| 8 | 6,6′-(1,2,4,5-tetrazine-3,6- | 37 | 80 |
| | diyl)dinicotinic acid | | |

Acidolysis of the enamide to the C-terminal α-amide was achieved for a range of acids. The reaction was shown efficient with strong acids. Cleavage of the enamide by the IEDDA reaction was further efficient with di-2-pyridyl substituted tetrazine, providing C-terminal α-amides in high yield.

### Example 3: Reaction Performance with Variation at Penultimate AA Position

The performance of the reaction with respect to the penultimate AA position of a example peptide was investigated for four sets of conditions. The general protocol outlined for screening scale α-amidation was used for these experiments.

Two photolabeling agents were selected for detailed analysis in the reaction, 3-Bromo-1H-pyrrole-2,5-dione (Chem. 1) and 4-chloro-7-nitrobenzofurazan (Chem. 2) which typically provided the best combination of cost, high conjugation yields, and facile photolysis under UV and visible light sources respectively. The final enamide cleavage step was performed with 5% v/v TFA for the acidolysis route, and with 3,6-Di-2-pyridyl-1,2,4,5-tetrazine for IEDDA mediated cleavage of the enamide. The general reaction conditions and results are presented in Tables 3 and 4 below.

TABLE 3
Reaction conditions A, B, C and D.

| Condi- | | | Enamide cleavage |
|---|---|---|---|
| tion | Photolabeling agent | Lightsource | reagent |
| A | 3-Bromo-1H-pyrrole-2,5- | 365 nm UV | 5% v/v TFA |
| | dione (Chem. 1) | light | |
| B | 3-Bromo-1H-pyrrole-2,5- | 365 nm UV | 3,6-Di-2-pyridyl- |
| | dione (Chem. 1) | light | 1,2,4,5-tetrazine |
| C | 4-chloro-7-nitrobenzofurazan | CFL or 450 | 5% v/v TFA |
| | (Chem. 2) | nm LEDs | |
| D | 4-chloro-7-nitrobenzofurazan | CFL or 450 | 3,6-Di-2-pyridyl- |
| | (Chem. 2) | nm LEDs | 1,2,4,5-tetrazine |

TABLE 4
Survey of reactivity of a generic peptide with varying penultimate amino acids, X,
under conditions A-D. The amino acid sequence of R2 (that is the sequence of the peptide
excluding the C-terminal cysteine amidation tag), IWTKDHEEVYEX of “generic peptide” is
displayed in the sequence list as SEQ ID NO 1.

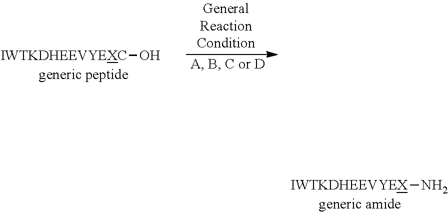

| | Amino acid, | Condition A, | Condition B, | Condition C, | Condition D, |
|---|---|---|---|---|---|
| Entry | X | yield [%] | yield [%] | yield [%] | yield [%] |
| 1 | Ala | 65 | 77 | 68 | 79 |
| 2 | Asp | 62 | 59 | 52 | 61 |
| 3 | Glu | 61 | 78 | 61 | 79 |
| 4 | Phe | 64 | 77 | 64 | 86 |
| 5 | Gly | 63 | 76 | 61 | 79 |
| 6 | His | 64 | 75 | 70 | 83 |
| 7 | Ile | 69 | 76 | 72 | 81 |
| 8 | Lys | 70 | 77 | 70 | 86 |
| 9 | Leu | 68 | 77 | 73 | 86 |
| 10 | Met | 60 | 66 | 66 | 80 |
| 11 | Asn | 72 | 76 | 72 | 82 |
| 12 | Pro | 78 | 74 | 83 | 86 |
| 13 | Gln | 60 | 79 | 58 | 83 |
| 14 | Arg | 69 | 76 | 71 | 85 |
| 15 | Ser | 54 | 78 | 57 | 83 |
| 16 | Thr | 44 | 77 | 48 | 78 |
| 17 | Val | 66 | 77 | 70 | 81 |
| 18 | Trp | 56 | 77 | 27 | 72 |
| 19 | Tyr | 59 | 77 | 56 | 84 |
| 20 | Ala* | 78 | 79 | 75 | 79 |

*The starting material sequence of Entry 20 varies from the sequence of generic peptide and
is displayed in the sequence list as SEQ. ID NO 3, and the sequence of the product of this
reaction is SEQ. ID NO 4.

The scope of the C-terminal α-amidation reaction was surveyed with respect to the penultimate amino acid position of a generic peptide. The reaction proceeds in good to excellent yields for any amino acid in the penultimate position, demonstrating a broad tolerance for side chain functionality at this position. The reaction proceeds in high efficiency when either 2-bromomaleimide or NBD-CI are utilized in the photolabeling step. High yields of the C-terminal α-amide are observed when the enamide is cleaved by acidolysis or by IEADDA reaction, allowing for choice between two complementary reactions that can be performed under acidic or neutral conditions, respectively.

### Example 4: Disulfide Containing Peptides

The present invention was used for the preparation of a peptide with a C-terminal α-amide which contained a disulfide bond. The protocol for batch scale reaction of peptides containing a disulfide bond identified above was used for this experiment following the reaction generally shown in Scheme 1, with the exception that no surfactant was used. A nucleophilic sulfide of cysteamine (8 equiv.) and an oxidizing partner of cystamine (1.2 equiv.) in H₂O was used. The cysteine rich backbone was globally alkylated with photolabel in the first step. Selective photolysis of the C-terminal alkylated cysteine occurred upon exposure to light. The remaining alkylated cysteines were removed with the nucleophilic sulfide and oxidized to liberate the cysteine sulfides and form the desired peptide containing a disulfide bond. The reaction then proceeded according to conditions outlined for enamide cleavage with TFA to afford the results presented in Table 5 below.

TABLE 5
C-terminal amidation yield of a disulfide
containing peptide subject to the
photochemical approach

| Polypeptide part (R2) | amide [%] |
|---|---|
| of starting material | yield |
| IECTKSEGCEEVYEADHGEP (SEQ ID NO. 15) | 60 |

Efficient amidation of the model peptide (IECTKSEGCEEVYEADHGEP, SEQ ID No. 15) which is to contain a disulfide bond in the amidated state demonstrates that peptides containing backbone cysteines readily undergo selective transformation to the desired C-terminal α-amide.

### Example 5: Preparation of Biologically Active Peptides

The present invention was used to synthesize biologically relevant peptides and marketed peptide therapeutics. The protocol outlined for screening scale α-amidation was used for these experiments, and the reaction generally followed Scheme 1 and conditions C or D as outlined in Table 3. The results are presented in Table 6.

TABLE 6
Preparation of biologically active
peptides according to invention.

| | | | amide | amide |
|---|---|---|---|---|
| | | | [%] | [%] |
| | Polypeptide part | | yield | yield |
| | (R2) of starting | Bioactive | Condition | Condition |
| Entry | material | peptide | C | D |
| 1 | HAEGTFTSDV SSYLEGQAAK | GLP-1 | 53 | 70 |
| | EFIAWLVKGR (SEQ. ID No. 3) | | | |
| 2 | PEGKGEQEHS QQKEEEEMAV | PSTi8 | 69 | 72 |
| | (SEQ. ID No. 4) | | | |
| 3 | IKPEAPGEDA SPEELNRYYA | pYY | n.d. | 70 |
| | SLRHYLNLVT RQRY | (3-36) | | |
| | (SEQ. ID No. 5) | | | |
| 4 | Pyr-HWSYGLRPG (SEQ. ID No. 6) | LHRH | n.d. | 78 |
| 5 | VPLPAGGGTV LTKMYPRGNH | Gastrin | 48 | 68 |
| | WAVGHLM (SEQ. ID No. 7) | releasing | | |
| | | peptide | | |
| 6 | SYSMEHFRWG KPVGKKRRPV KVYP | Cosyntropin | n.d. | 63 |
| | (SEQ. ID No. 8) | | | |
| 7 | QDEGSEATGF LPAAGEKTSG | QRF-amide | n.d. | 73 |
| | PLGNLAEELN GYSRKKGGFS FRF | | | |
| | (SEQ. ID No. 9) | | | |
| 8 | HGEGTFTSDL SKQMEEEAVR | EP45 | 56 | 49 |
| | LFIEWLKNGG PSSRHYLNLV TRQRY | | | |
| | (SEQ. ID No. 10) | | | |
| 9 | Myristic acid-GTNLSVPNPLG | Bulevirtide | n.d. | 78 |
| | FFPDHQLDPA FGANSNNPDW | | | |
| | DFNPNKDHWP EANKVG | | | |
| | (SEQ. ID No. 11) | | | |
| 10 | SFSGFGSPLD RLSAGSVDHK | osteocrin | 57 | 66 |
| | GKQRKVVDHP KRRFGIPMDR | | | |
| | IGRNRLSNSR (SEQ. ID No. 12) | | | |
| 11 | Ac-YTSLIHSLIE ESQNQQEKNE | Enfuvirtide | 38 | 49 |
| | QELLELDKWA SLWNWF | | | |
| | (SEQ. ID No. 13) | | | |
| 12 | KCNTATCATQRLANFLVHSSNNFGPILP | Pramlintide | 28 | n.d. |
| | PTNVGSNTY (SEQ ID No. 14) | | | |

The C-terminal cysteine extended precursors of biologically active peptides and marketed peptide therapeutics were prepared and subjected to the C-terminal α-amidation process. Good yields of the corresponding biologically relevant C-terminal α-amides were obtained in all examples, highlighting the mild reaction conditions and broad scope of the invention. The C-terminal α-amidation process performed well regardless of the size and composition of the peptide, demonstrating the applicability of the chemistry to any peptide or protein.

### Example 6: Upscaled Preparation of GLP-1 Receptor-Amylin Receptor Co-Agonist Intermediate

The present invention was applied on a larger scale using a hybrid batch—photoflow procedure described in detail below.

The present example was done on a peptide R2 with an amino acid sequence as set out in SEQ ID No. 17, which comprises an amino acid sequence as set out in SEQ ID No. 16 plus an N-terminal extension having an amino acid sequence as set out in SEQ ID No. 18.

To a one-litre glass reactor wrapped in aluminium foil under nitrogen atmosphere, and containing a solution of peptide R2 according to SEQ ID No. 17 with a C-terminal cysteine amidation tag (12.4 g, 1.44 mmol) in aqueous 0.2-0.3 mM TCEP (780 ml, pH 7), was added a solution of NBD-CI (519 mg, 2.6 mmol) in MeCN (5.2 ml). After stirring at ambient temperature for 80 minutes, HPLC analysis indicated reaction completion.

The resulting solution was subjected to irradiation at 405 nm (blue LEDs, input power of 61 W) in a Corning™ Lab Photo Reactor (2.7 mL fluidic module volume, 10 mL/min flow rate, 20° C. reactor heat exchange temperature), and the reactor was flushed with an additional 50 ml water. HPLC analysis indicated a consumption of at least 80% of the starting material. 6M phosphoric acid (H₃PO₄, 160 mL) was added to the resulting solution. After stirring for 16 hours, L-histidine (7.45 g) was added and the mixture was stirred at ambient temperature for 10 minutes. 6M of aqueous KOH solution was then added carefully until the solution attained pH 8.5 while maintaining a temperature below 40° C. The resulting mixture was concentrated by ultrafiltration (MWCO 5 kDa) to 1.0 L and diafiltrated (MWCO 5 kDa) with 3.0 L of 20 mM Tris buffer pH 8 while maintaining a constant retentate volume (continuous mode) between 0.8 and 1 L. The final mixture (800 mL) was analyzed by HPLC. The conversion results are indicated below.

TABLE 7
C-terminal amidation yield of an upscaled
procedure for a peptide having an N-
terminal extension

| Polypeptide part (R2) | amide [%] |
|---|---|
| of starting material | yield |
| DVKPGQPLEDEGDREGTFTSDV | 78 |
| SSYLEEQAAREFIAWLVRGRKG | |
| GGGEASELSTAALGRLSAELHE | |
| LATLPRTETGSGSP | |
| (SEQ ID No. 17) | |

Reaction samples were diluted 10-fold in 1:1 v/v MeCN/water and analyzed by RP-HPLC analysis (Kinetex 1.7 μm C18 100 Å 100×2.1 mm, eluent A: 10% MeCN+0.1% TFA in water, eluent B: 90% MeCN+0.1% TFA in water. Gradient: 20 to 50% B in 12 min, UV detection at both 214 nm and 420 nm. Conversion yields were calculated using an external HPLC standard.

This example shows a larger scale production of a peptide with C-terminal amide where the respective peptide amide was produced in high yield using the photochemical C-terminal α-amidation method. Additionally, this example shows that the photochemical method can be completed in a hybrid setup, where only the photochemical conversion step (step (b) of scheme 1 or 2) was performed in a flow set-up. The example in general highlights the robustness and scalability of the method.

While certain features of the invention have been illustrated and described herein, many modifications, substitutions, changes, and equivalents will now occur to those of ordinary skill in the art. It is, therefore, to be understood that the appended embodiments are intended to cover all such modifications and changes as fall within the true spirit of the invention.

1. A method for producing a peptide or protein comprising a C-terminal α-amide of formula IV, following Scheme 1 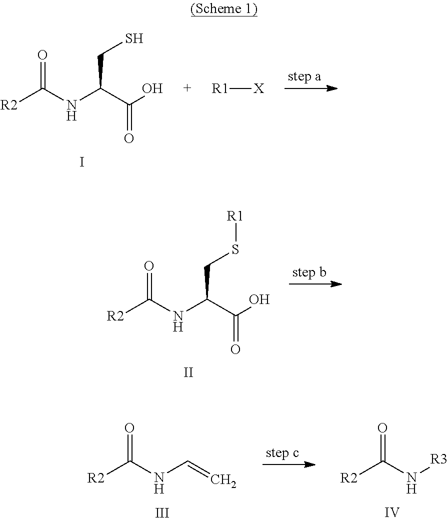
wherein R2 is a polypeptide,
R1-X is a photolabeling agent, wherein R1 is a photolabel and X is a leaving group,
R3 is selected from the group consisting of hydrogen, methyl and ethyl,
comprising the steps of
Step (a). coupling a peptide or protein comprising a C-terminal cysteine amidation tag of formula I with the photolabel (R1) to obtain a peptide-photolabel conjugate of formula II;
Step (b). irraditating the peptide-photolabel conjugate of formula II to obtain a C-terminal enamide of formula III via a photochemical conversion; and
Step (c). cleaving the resulting C-terminal enamide of formula III to obtain the C-terminal α-amide of formula IV.

2. The method according to claim 1, wherein R3 is hydrogen.

3. The method according to claim 1, wherein R2 comprises an amino acid sequence as set out in any one of SEQ. ID NO.: 3, 4, 5, 6, 7, 8, 9, 10, 11, 12, 13, 14, 16, 17.

4. The method according to claim 1, wherein R2 is an amino acid sequence as set out in any one of SEQ. ID NO. 3-14 and 16-17.

5. The method according to claim 1, wherein the photolabeling agent (R1-X) is selected from the group consisting of 3-Bromo-1H-pyrrole-2,5-dione, 4-chloro-7-nitrobenzofurazan, 2-bromo-1,4-naphthoquinone, 1-fluoro-2,4-dinitrobenzene and 4-fluoro-7-sulfamoylbenzofurazan.

6. The method according to claim 1, wherein the photolabeling agent (R1-X) is 3-Bromo-1H-pyrrole-2,5-dione (Chem. 1) or 4-chloro-7-nitrobenzofurazan (Chem. 2):

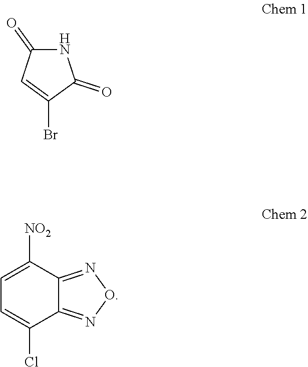 [SMILES: O=C1C=C(Br)C(=O)N1 ; O=[N+]([O-])c1ccc(Cl)c2nonc12]

7. The method according to claim 1, wherein the photolabeling agent is 3-Bromo-1H-pyrrole-2,5-dione (Chem. 1):

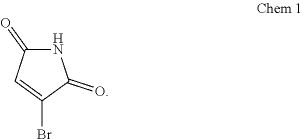

8. The method according to claim 1, wherein the photolabeling agent is 4-chloro-7-nitrobenzofurazan (Chem. 2):

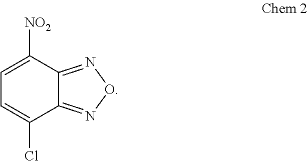 [SMILES: O=[N+]([O-])c1ccc(Cl)c2nonc12]

9. The method according to claim 1, wherein step (a) of the method takes place in an aqueous reaction buffer, the aqueous buffer selected from the group consisting of bis-tris methane, tris, triethanolamine and phosphate.

10. The method according to claim 1, wherein a source of light for irradiating the peptide-photolabel conjugate has a wavelength between 365-500 nm.

11. The method according to claim 1, wherein at least part of the method is performed in a flow reactor.

12. The method according to claim 1, further comprising the steps of
coupling a cysteine of the peptide R2 with the photolabel R1 forming a photolabel-protected cysteine prior to step (b); and
releasing the photolabel-protected cysteine from the peptide R2 after step (b).

13. The method according to claim 12, wherein a nucleophilic sulfide and an oxidation partner are provided.

14. A method for producing a peptide or protein comprising a C-terminal α-amide of formula IV following Scheme 2, 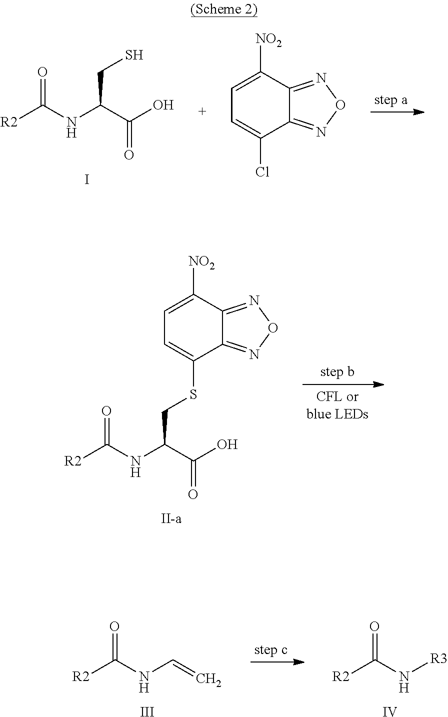
wherein R2 is a polypeptide comprising an amino acid sequence as set out in SEQ ID No. 16,
R3 is hydrogen,
comprising the steps of
Step (a). coupling a peptide or protein comprising a C-terminal cysteine amidation tag of formula I with 4-chloro-7-nitrobenzofurpazan to obtain a peptide-photolabel conjugate of formula II-a;
Step (b). irradiating the peptide-photolabel conjugate of formula II-a with light having a wavelength between 400-450 nm to obtain a C-terminal enamide of formula III via photochemical conversion; and
Step (c). cleaving the C-terminal enamide of formula III to obtain the C-terminal α-amide of formula IV.

15. (canceled)

16. A pharmaceutical composition comprising the peptide or protein produced according to claim 1.

17. A pharmaceutical composition comprising the peptide or protein produced according to claim 14.
